(19) United States
(12) Patent Application Publication
(10) Pub. No.: US 2024/0090808 A1
(43) Pub. Date: Mar. 21, 2024

(54) MULTI-SENSORY EAR-WORN DEVICES FOR STRESS AND ANXIETY DETECTION AND ALLEVIATION

(71) Applicant: Starkey Laboratories, Inc., Eden Prairie, MN (US)

(72) Inventors: Achintya Kumar Bhowmik, Cupertino, CA (US); Adrian Lister, Victoria (CA); Christophe Micheyl, Lyon (FR); Paul N. Reinhart, Minneapolis, MN (US); Justin R. Burwinkel, Eden Prairie, MN (US); Krishna Chaithanya Vastare, Eden Prairie, MN (US); Gerard N. Weisensel, Savage, MN (US)

(21) Appl. No.: 18/275,861

(22) PCT Filed: Feb. 4, 2022
(86) PCT No.: PCT/US2022/015304

§ 371 Date: Aug. 4, 2023

Related U.S. Application Data
(60) Provisional application No. 63/146,501, filed on Feb. 5, 2021.

(51) Int. Cl. A61B 5/16; A61B 5/00; G10L 25/63

(52) U.S. Cl. CPC A61B 5/165; A61B 5/0002; A61B 5/4803; A61B 5/486; A61B 5/6817; A61B 5/7267; A61B 5/7405; G10L 25/63; A61B 5/02405; A61B 2560/02; A61B 2562/0204; A61B 2562/0219

(57) ABSTRACT

Embodiments herein relate to ear-wearable stress and anxiety monitoring systems, devices and methods. Embodiments herein further relate to ear-wearable systems and devices that can detect and take actions to alleviate device wearer's stress and anxiety. In an embodiment an ear-wearable stress and/or anxiety monitoring system is included having a control circuit, a microphone, and a sensor package that can include a motion sensor. The ear-wearable system is configured to evaluate data from at least one of the microphone and the sensor package and classify a stress level of a device wearer using a machine learning classification model and periodically update the machine learning classification model based on indicators of stress experienced by the device wearer.

This application is being filed as a PCT International Patent application on Feb. 4, 2022 in the name of Starkey Laboratories, Inc., a U.S. national corporation, applicant for the designation of all countries, and Achintya Kumar Bhowmik, a U.S. Citizen, applicant and inventor for the designation of all countries, and Adrian Lister, a Canadian Citizen, and Christophe Micheyl, a French Citizen, and Paul N. Reinhart, a U.S. Citizen, and Justin R. Burwinkel, a U.S. Citizen, and Krishna Chaithanya Vastare, an Indian Citizen, and Gerard N. Weisensel, a U.S. Citizen, inventor(s) for the designation of all countries, and claims priority to U.S. Provisional Patent Application No. 63/146,501 filed Feb. 5, 2021, the contents of which are herein incorporated by reference in its entirety.

### FIELD

Embodiments herein relate to ear-wearable stress and anxiety monitoring systems, devices and methods. Embodiments herein further relate to ear-wearable systems and devices that can detect and take actions to alleviate device wearer's stress and anxiety.

### BACKGROUND

It is well known that stress and anxiety can adversely affect human health. As one example, studies have shown that stress and anxiety have many adverse effects on the human nervous system and can even cause structural changes in different parts of the brain. In particular, chronic stress can lead to atrophy of brain mass and decrease its weight. These structural changes bring about long-term adverse effects on the nervous and cognition system.

As another example, stress has many adverse effects on the immune system. Generally, people under stress are more likely to have an impaired immune system and, as a result, suffer from more frequent illness.

In addition, stress, whether acute or chronic, can have a negative effect on the function of the cardiovascular system. Acute stress leads to activation of the sympathetic nervous system, which results in an increase in heart rate, strength of contraction, vasodilation in the arteries of skeletal muscles, a narrowing of the veins, contraction of the arteries in the spleen and kidneys, and decreased sodium excretion by the kidneys. Chronic stress leads to an increased risk of cardiac disease, thrombosis and ischemia, as well as increased platelet aggregation and stroke. Anxiety causes the release of stress hormones in the body that can trigger an increase in the heart rate and a narrowing of the blood vessels. Both changes can cause blood pressure to rise significantly.

Stress also adversely impacts the gastrointestinal (GI) system. For example, stress can affect the absorption process, intestinal permeability, mucus and stomach acid secretion, function of ion channels, and GI inflammation. Stress also increases the response of the GI system to inflammation and may reactivate previous inflammation and accelerate the inflammation process by secretion of immuno-mediators.

Also, there is a broad relationship between stress and the endocrine system. Stress can activate, or otherwise change the activity of, many endocrine processes associated with the hypothalamus, pituitary and adrenal glands, the adrenergic system, gonads, thyroid, and the pancreas.

Stress can also negatively impact the auditory system. For example, stress can impact tinnitus, contributing to causation and/or worsening of the condition.

As with many things that impact human health, early detection and intervention are critical for optimally preventing the adverse effects of stress and anxiety on health. However, current procedures to determine the onset and progress of stress and anxiety are mostly subjective methods including questionnaires such as the perceived stress scale (PSS) administered and evaluated by professionals. Unfortunately, these methods are generally not conducive to the early detection of stress and stress related disorders. Further, these methods have limited accuracy as they are highly dependent on subjective inputs from the individuals answering the questionnaires.

### SUMMARY

Embodiments herein relate to ear-wearable stress and anxiety monitoring systems, devices and methods. Embodiments herein further relate to ear-wearable systems and devices that can detect and take actions to alleviate device wearer's stress and anxiety. In a first aspect, an ear-wearable stress and/or anxiety monitoring system is included having a control circuit, a microphone, and a sensor package that can include a motion sensor. The ear-wearable system is configured to evaluate data from at least one of the microphone and the sensor package and classify a stress level of a device wearer using a machine learning classification model and periodically update the machine learning classification model based on indicators of stress experienced by the device wearer.

In a second aspect, in addition to one or more of the preceding or following aspects, or in the alternative to some aspects, the indicators of stress experienced by the device wearer are derived from data produced by at least one of the microphone and the sensor package.

In a third aspect, in addition to one or more of the preceding or following aspects, or in the alternative to some aspects, the indicators of stress experienced by the device wearer are derived from inputs provided by the device wearer.

In a fourth aspect, in addition to one or more of the preceding or following aspects, or in the alternative to some aspects, the inputs provided by the device wearer include a tap.

In a fifth aspect, in addition to one or more of the preceding or following aspects, or in the alternative to some aspects, the inputs provided by the device wearer include a head gesture.

In a sixth aspect, in addition to one or more of the preceding or following aspects, or in the alternative to some aspects, the inputs provided by the device wearer include a response to a query.

In a seventh aspect, in addition to one or more of the preceding or following aspects, or in the alternative to some aspects, the indicators of stress experienced by the device wearer are received by the ear-wearable system from an external source. In an eighth aspect, in addition to one or more of the preceding or following aspects, or in the alternative to some aspects, the ear-wearable system is configured to correlate possible detected stressors with subsequent classified stress levels of the device wearer.

In a ninth aspect, in addition to one or more of the preceding or following aspects, or in the alternative to some aspects, the ear-wearable system is configured to weight certain possible detected stressors in the machine learning classification model more heavily based on the correlation.

In a tenth aspect, in addition to one or more of the preceding or following aspects, or in the alternative to some aspects, data from the microphone is processed to distinguish a voice of the device wearer from a voice of a third party.

In an eleventh aspect, in addition to one or more of the preceding or following aspects, or in the alternative to some aspects, the voice of the device wearer is treated as an indicator of stress experienced by the device wearer.

In a twelfth aspect, in addition to one or more of the preceding or following aspects, or in the alternative to some aspects, the voice of the third party is treated as a possible detected stressor.

In a thirteenth aspect, in addition to one or more of the preceding or following aspects, or in the alternative to some aspects, the ear-wearable system is configured to correlate sleep of the device wearer with subsequent classified stress levels of the device wearer.

In a fourteenth aspect, in addition to one or more of the preceding or following aspects, or in the alternative to some aspects, the ear-wearable system is configured to detect meals and correlate classified stress levels with detected meals.

In a fifteenth aspect, in addition to one or more of the preceding or following aspects, or in the alternative to some aspects, the ear-wearable system is configured to correlate stress levels with a time since a last meal.

In a sixteenth aspect, in addition to one or more of the preceding or following aspects, or in the alternative to some aspects, the ear-wearable system is configured to detect displacement behaviors and correlate classified stress levels with detected displacement behaviors.

In a seventeenth aspect, in addition to one or more of the preceding or following aspects, or in the alternative to some aspects, the sensor package can include at least one selected from the group consisting of a heart rate sensor, a heart rate variability sensor, an electrocardiogram (ECG) sensor, a blood oxygen sensor, a blood pressure sensor, a skin conductance sensor, a photoplethysmography (PPG) sensor, an electromyography (EMG) sensor, a core body temperature sensor, a motion sensor, an electroencephalograph (EEG) sensor, and a respiratory sensor.

In an eighteenth aspect, in addition to one or more of the preceding or following aspects, or in the alternative to some aspects, the motion sensor includes at least one of an accelerometer and a gyroscope.

In a nineteenth aspect, in addition to one or more of the preceding or following aspects, or in the alternative to some aspects, the ear-wearable system is configured to send information regarding classified stress levels to an electronic medical record system.

In a twentieth aspect, in addition to one or more of the preceding or following aspects, or in the alternative to some aspects, the ear-wearable system is configured to send information regarding classified stress levels to a third party.

In a twenty-first aspect, in addition to one or more of the preceding or following aspects, or in the alternative to some aspects, the ear-wearable system is configured to distinguish between acute and chronic stress of the device wearer.

In a twenty-second aspect, in addition to one or more of the preceding or following aspects, or in the alternative to some aspects, the ear-wearable system is configured to classify detected stress as acute stress, chronic stress, or both.

In a twenty-third aspect, a method of monitoring stress of an individual using an ear-wearable stress and/or anxiety monitoring system is included, the method including evaluating data from at least one of a microphone and a sensor package, classifying a stress level of the individual using a machine learning classification model, and updating the machine learning classification model based on indicators of stress experienced by the individual.

In a twenty-fourth aspect, in addition to one or more of the preceding or following aspects, or in the alternative to some aspects, the method can further include deriving indicators of stress experienced by the individual from data produced by at least one of the microphone and the sensor package.

In a twenty-fifth aspect, in addition to one or more of the preceding or following aspects, or in the alternative to some aspects, the method can further include deriving indicators of stress experienced by the individual from input provided by the individual.

In a twenty-sixth aspect, in addition to one or more of the preceding or following aspects, or in the alternative to some aspects, the method can further include deriving indicators of stress experienced by the individual from input provided by an external device.

In a twenty-seventh aspect, in addition to one or more of the preceding or following aspects, or in the alternative to some aspects, the method can further include correlating possible detected stressors with subsequent classified stress levels of the individual.

In a twenty-eighth aspect, in addition to one or more of the preceding or following aspects, or in the alternative to some aspects, wherein correlating possible detected stressors with subsequent classified stress levels of the individual further includes weighting certain possible detected stressors in the machine learning classification model more heavily based on the correlation.

In a twenty-ninth aspect, in addition to one or more of the preceding or following aspects, or in the alternative to some aspects, the method can further include processing data from the microphone to distinguish a voice of the individual from a voice of a third party.

In a thirtieth aspect, in addition to one or more of the preceding or following aspects, or in the alternative to some aspects, the method can further include detecting meals of the individual and correlating classified stress levels with detected meals.

In a thirty-first aspect, in addition to one or more of the preceding or following aspects, or in the alternative to some aspects, the method can further include detecting displacement behaviors and correlating classified stress levels with detected displacement behaviors.

In a thirty-second aspect, in addition to one or more of the preceding or following aspects, or in the alternative to some aspects, the method can further include sending information regarding classified stress levels to an electronic medical record system.

In a thirty-third aspect, in addition to one or more of the preceding or following aspects, or in the alternative to some aspects, the method can further include sending information regarding classified stress levels to a third party.

In a thirty-fourth aspect, in addition to one or more of the preceding or following aspects, or in the alternative to some aspects, the method can further include distinguishing between acute and chronic stress of the individual.

In a thirty-fifth aspect, an ear-wearable stress and/or anxiety monitoring system is included having a control circuit, a microphone in electrical communication with the control circuit, an electroacoustic transducer configured for placement within an ear canal of a device wearer, and a sensor package including a motion sensor, wherein the ear-wearable system is configured to evaluate data from at least one of the microphone and the sensor package and classify a device wearer's stress level using a machine learning classification model, and provide information to the device wearer relating to the classified stress level in a discrete manner.

In a thirty-sixth aspect, in addition to one or more of the preceding or following aspects, or in the alternative to some aspects, the information relating to the classified stress level is provided through the electroacoustic transducer.

In a thirty-seventh aspect, in addition to one or more of the preceding or following aspects, or in the alternative to some aspects, the information provided to the device wearer relating the classified stress level includes verbal information.

In a thirty-eighth aspect, in addition to one or more of the preceding or following aspects, or in the alternative to some aspects, the information provided to the device wearer relating the classified stress level includes non-verbal sound.

In a thirty-ninth aspect, in addition to one or more of the preceding or following aspects, or in the alternative to some aspects, the non-verbal sound includes music.

In a fortieth aspect, in addition to one or more of the preceding or following aspects, or in the alternative to some aspects, the information relating to the classified stress level includes instructions for an action for the device wearer to take to reduce the stress level of the device wearer.

In a forty-first aspect, in addition to one or more of the preceding or following aspects, or in the alternative to some aspects, the instructions include breathing instructions.

In a forty-second aspect, in addition to one or more of the preceding or following aspects, or in the alternative to some aspects, the breathing instructions include a suggestion to consume a food item and/or take a medication.

In a forty-third aspect, in addition to one or more of the preceding or following aspects, or in the alternative to some aspects, the information relating to the classified stress level includes a sound preselected by the device wearer.

In a forty-fourth aspect, in addition to one or more of the preceding or following aspects, or in the alternative to some aspects, the information relating to the classified stress level includes information regarding a predicted stress level of the device wearer in a subsequent time period.

In a forty-fifth aspect, in addition to one or more of the preceding or following aspects, or in the alternative to some aspects, the ear-wearable system is configured to periodically update the machine learning classification model based on indicators of stress experienced by the device wearer.

In a forty-sixth aspect, in addition to one or more of the preceding or following aspects, or in the alternative to some aspects, further can include a power supply circuit.

In a forty-seventh aspect, in addition to one or more of the preceding or following aspects, or in the alternative to some aspects, the ear-wearable system is configured to send information regarding classified stress levels to an electronic medical record system.

In a forty-eighth aspect, in addition to one or more of the preceding or following aspects, or in the alternative to some aspects, the ear-wearable system is configured to send information regarding classified stress levels to a third party.

In a forty-ninth aspect, in addition to one or more of the preceding or following aspects, or in the alternative to some aspects, the ear-wearable system is configured to cross-reference classified stress levels against a calendar of the device wearer.

In a fiftieth aspect, in addition to one or more of the preceding or following aspects, or in the alternative to some aspects, the ear-wearable system is configured to distinguish between acute and chronic stress of the device wearer.

In a fifty-first aspect, in addition to one or more of the preceding or following aspects, or in the alternative to some aspects, the ear-wearable system is configured to detect meals of the device wearer and cross-reference classified stress levels against detected meal times.

In a fifty-second aspect, a method of monitoring stress of an individual using an ear-wearable stress and/or anxiety monitoring system is included, the method including evaluating data from at least one of a microphone and a sensor package, classifying a stress level of the individual using a machine learning classification model, and providing information to the individual relating to the classified stress level in a discrete manner.

In a fifty-third aspect, in addition to one or more of the preceding or following aspects, or in the alternative to some aspects, the method can further include providing information to the individual relating to the classified stress level through an electroacoustic transducer in the ear canal of the individual.

In a fifty-fourth aspect, in addition to one or more of the preceding or following aspects, or in the alternative to some aspects, the method can further include providing instructions to the individual regarding an action to take to reduce the stress level of the individual.

In a fifty-fifth aspect, in addition to one or more of the preceding or following aspects, or in the alternative to some aspects, the instructions to the individual include breathing instructions.

In a fifty-sixth aspect, in addition to one or more of the preceding or following aspects, or in the alternative to some aspects, the instructions to the individual include a suggestion to consume a food item and/or take a medication.

In a fifty-seventh aspect, in addition to one or more of the preceding or following aspects, or in the alternative to some aspects, wherein information to the individual relating to the classified stress level includes a sound preselected by the device wearer.

In a fifty-eighth aspect, in addition to one or more of the preceding or following aspects, or in the alternative to some aspects, wherein information to the individual relating to the classified stress level includes information regarding a predicted stress level of the device wearer in a subsequent time period.

In a fifty-ninth aspect, in addition to one or more of the preceding or following aspects, or in the alternative to some aspects, the method can further include deriving indicators of stress experienced by the individual from input provided by an external device.

In a sixtieth aspect, in addition to one or more of the preceding or following aspects, or in the alternative to some aspects, the method can further include correlating possible detected stressors with subsequent classified stress levels of the individual.

In a sixty-first aspect, in addition to one or more of the preceding or following aspects, or in the alternative to some aspects, wherein correlating possible detected stressors with subsequent classified stress levels of the individual further includes weighting certain possible detected stressors in the machine learning classification model more heavily based on the correlation.

In a sixty-second aspect, in addition to one or more of the preceding or following aspects, or in the alternative to some aspects, the method can further include processing data from the microphone to distinguish a voice of the individual from a voice of a third party.

In a sixty-third aspect, in addition to one or more of the preceding or following aspects, or in the alternative to some aspects, the method can further include detecting meals of the individual and correlating classified stress levels with detected meals.

In a sixty-fourth aspect, in addition to one or more of the preceding or following aspects, or in the alternative to some aspects, the method can further include detecting displacement behaviors and correlating classified stress levels with detected displacement behaviors.

In a sixty-fifth aspect, in addition to one or more of the preceding or following aspects, or in the alternative to some aspects, the method can further include sending information regarding classified stress levels to an electronic medical record system.

In a sixty-sixth aspect, in addition to one or more of the preceding or following aspects, or in the alternative to some aspects, the method can further include sending information regarding classified stress levels to a third party.

In a sixty-seventh aspect, in addition to one or more of the preceding or following aspects, or in the alternative to some aspects, the method can further include distinguishing between acute and chronic stress of the individual.

In a sixty-eighth aspect, an ear-wearable stress and/or anxiety monitoring system is included having a control circuit, a microphone in electrical communication with the control circuit, an electroacoustic transducer configured for placement within an ear canal of a device wearer, and a sensor package including a motion sensor, wherein the ear-wearable system is configured to detect an occurrence of stress of the device wearer exceeding a threshold value, and evaluate data from at least one of the microphone and the sensor package over a lookback period to detect a trigger or contributing cause of the stress.

In a sixty-ninth aspect, in addition to one or more of the preceding or following aspects, or in the alternative to some aspects, the ear-wearable system is configured to execute an operation to remediate the trigger or contributing cause of the stress.

In a seventieth aspect, in addition to one or more of the preceding or following aspects, or in the alternative to some aspects, the operation includes reducing the volume of sounds exceeding a threshold value.

In a seventy-first aspect, in addition to one or more of the preceding or following aspects, or in the alternative to some aspects, the volume is reduced by actuating an autovent.

In a seventy-second aspect, in addition to one or more of the preceding or following aspects, or in the alternative to some aspects, the volume is reduced by adjusting a gain value of the ear-wearable system.

In a seventy-third aspect, in addition to one or more of the preceding or following aspects, or in the alternative to some aspects, the ear-wearable system is further configured to characterize the stress causing potential of ambient sound.

In a seventy-fourth aspect, in addition to one or more of the preceding or following aspects, or in the alternative to some aspects, the ear-wearable system is configured to provide instructions regarding recommended interventions to the device wearer.

In a seventy-fifth aspect, in addition to one or more of the preceding or following aspects, or in the alternative to some aspects, the ear-wearable system is configured to record data regarding occurrences of stress and calculate a trend regarding the same.

In a seventy-sixth aspect, in addition to one or more of the preceding or following aspects, or in the alternative to some aspects, the ear-wearable system is configured to change instructions regarding recommended interventions if the trend is negative.

In a seventy-seventh aspect, in addition to one or more of the preceding or following aspects, or in the alternative to some aspects, the ear-wearable system is configured to predict a future occurrence of stress of the device wearer based on at least one of current inputs from the microphone and/or sensor package, information from a calendar of the device wearer, and a geographic location of the device wearer.

In a seventy-eighth aspect, in addition to one or more of the preceding or following aspects, or in the alternative to some aspects, the ear-wearable system is configured to distinguish between acute and chronic stress of the device wearer.

In a seventy-ninth aspect, in addition to one or more of the preceding or following aspects, or in the alternative to some aspects, the ear-wearable system is configured to evaluate data from the microphone to identify a voice of an individual serving as a trigger of the stress.

In an eightieth aspect, in addition to one or more of the preceding or following aspects, or in the alternative to some aspects, the ear-wearable system is configured to evaluate data received from a personal electronic device of a third person to identify an individual serving as a trigger of the stress.

In an eighty-first aspect, a method of monitoring stress and/or anxiety of an individual using an ear-wearable stress monitoring system is included, the method including detecting an occurrence of stress of the device wearer exceeding a threshold value and evaluating data from at least one of a microphone and a sensor package over a lookback period to detect a trigger of the stress.

In an eighty-second aspect, in addition to one or more of the preceding or following aspects, or in the alternative to some aspects, the method can further include executing an operation to remediate the trigger of the stress.

In an eighty-third aspect, in addition to one or more of the preceding or following aspects, or in the alternative to some aspects, the operation includes reducing the volume of sounds exceeding a threshold value.

In an eighty-fourth aspect, in addition to one or more of the preceding or following aspects, or in the alternative to some aspects, the volume is reduced by adjusting a gain value.

In an eighty-fifth aspect, in addition to one or more of the preceding or following aspects, or in the alternative to some aspects, the volume is decreased by actuating an autovent.

In an eighty-sixth aspect, in addition to one or more of the preceding or following aspects, or in the alternative to some aspects, the method can further include providing information to the individual relating to the classified stress level through an electroacoustic transducer in the ear canal of the individual.

In an eighty-seventh aspect, in addition to one or more of the preceding or following aspects, or in the alternative to some aspects, the method can further include providing instructions to the individual regarding an action to take related to the detected trigger of the stress.

In an eighty-eighth aspect, in addition to one or more of the preceding or following aspects, or in the alternative to some aspects, the instructions to the individual can include breathing instructions.

In an eighty-ninth aspect, in addition to one or more of the preceding or following aspects, or in the alternative to some aspects, the instructions to the individual can include a suggestion to consume a food item and/or take a medication.

In a ninetieth aspect, in addition to one or more of the preceding or following aspects, or in the alternative to some aspects, the method can further include deriving indicators of stress experienced by the individual from input provided by an external device.

In a ninety-first aspect, in addition to one or more of the preceding or following aspects, or in the alternative to some aspects, the method can further include correlating possible detected stressors with subsequent classified stress levels of the individual.

In a ninety-second aspect, in addition to one or more of the preceding or following aspects, or in the alternative to some aspects, wherein correlating possible detected stressors with subsequent classified stress levels of the individual further includes weighting certain possible detected stressors in the machine learning classification model more heavily based on the correlation.

In a ninety-third aspect, in addition to one or more of the preceding or following aspects, or in the alternative to some aspects, the method can further include processing data from a microphone to distinguish a voice of the individual from a voice of a third party.

In a ninety-fourth aspect, in addition to one or more of the preceding or following aspects, or in the alternative to some aspects, the method can further include sending information regarding detected stress triggers to an electronic medical record system.

In a ninety-fifth aspect, in addition to one or more of the preceding or following aspects, or in the alternative to some aspects, the method can further include sending information regarding detected stress triggers to a third party.

In a ninety-sixth aspect, in addition to one or more of the preceding or following aspects, or in the alternative to some aspects, the method can further include identifying a voice of another individual serving as a detected stress trigger.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of indicators of stress and anxiety in accordance with various embodiments herein.

FIG. 2 is a schematic view of stressors and items related to stress and anxiety in accordance with various embodiments herein.

FIG. 3 is a schematic view of an ear-wearable device in accordance with various embodiments herein.

FIG. 4 is a schematic view of an ear-wearable device within the ear in accordance with various embodiments herein.

FIG. 5 is a schematic view of classification models in accordance with various embodiments herein.

FIG. 6 is a schematic view of classification models in accordance with various embodiments herein.

FIG. 7 is a schematic view of an ear-wearable stress monitoring system in accordance with various embodiments herein.

FIG. 8 is a schematic view of an ear-wearable device in accordance with various embodiments herein.

FIG. 9 is a schematic view of an accessory device in accordance with various embodiments herein.

FIG. 10 is a schematic view of an ear-wearable device in accordance with various embodiments herein.

FIG. 11 is a schematic view of an ear-wearable device in accordance with various embodiments herein.

FIG. 12 is a schematic view of an ear-wearable device in accordance with various embodiments herein.

FIG. 13 is a schematic view of an ear-wearable device in accordance with various embodiments herein.

FIG. 14 is a schematic representation of possible stressors and detected stress along a timeline.

FIG. 15 is a block diagram view of components of an ear-wearable device in accordance with various embodiments herein.

FIG. 16 is a block diagram view of components of an accessory device in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

As described above, stress and anxiety can contribute to or cause many different health problems and impacts a vast array of bodily systems including the nervous system, the immune system, the cardiovascular system, the GI system, and the endocrine system. Existing techniques for measuring stress and anxiety are largely subjective, imprecise, and only available in certain settings. However, embodiments herein can provide for systems and devices that can accurately measure stress and anxiety levels for individuals in their daily routines in real-life situations rather than in specialized laboratory settings.

Ear-wearable devices herein can function as multifunctional health devices that not only amplify sound to compensate for the wearer's hearing deficiencies and/or provide treatment for conditions such as tinnitus, but also measure multiple physiological parameters using embedded sensors. Systems and devices herein can apply algorithms to derive health insights related to stress from these data, including applying advanced machine learning techniques that are trained and validated with ground-truth data from representative groups of patients. Besides helping people hear better and tracking their physical health, the multi-sensory ear-worn devices herein can remotely and continuously detect, monitor, and alleviate stress and anxiety.

In various embodiments, the devices herein incorporate built-in sensors for measuring and analyzing multiple physiological and cognitive data, including, but not limited to, core body temperature, cardiovascular parameters, including heart rate and heart rate variability, blood oxygen saturation (SpO2), blood pressure, electrodermal activities such as skin conductance determined from skin galvanic responses, physical activities derived from accelerometers and gyroscopes, vocal biomarkers extracted from voice, respiratory events and rates, electroencephalographs (EEG) and electrocardiograms (ECG). Data from these sensors, amongst other data utilized as described herein, can be processed by devices and systems herein to accurately gauge levels of stress and anxiety experienced by device wearers.

Machine learning models utilized herein are developed and trained with patient data, and deployed for on-device monitoring, classification, and communication, taking advantage of the fact that such ear-wearable devices will be continuously worn by the hearing-impaired patients. In various embodiments, upon detection of the onset of stress and anxiety, and in some cases the assessment of the progression of the conditions, alleviations can be provided via appropriate channels including, for example, acoustic signals and instructions for remedial steps via the speakers embedded within the in-ear devices.

Further, recognizing that physiological responses to the onset and levels of stress and anxiety vary from person to person, as well as the fact that reactions to possible stressors vary highly amongst individuals, embodiments herein can include an architecture for personalization via on-device in-situ training and optimization phase(s).

In various embodiments, an ear-wearable stress monitoring system herein can include a control circuit, a microphone, and a sensor package. The sensor package can include various sensors such as a motion sensor, as well as other sensors described herein. The ear-wearable stress monitoring system can be configured to evaluate data from at least one of the microphone and a sensor that is part of the sensor package and classify a stress level of a device wearer using a machine learning classification model. In various embodiments, the system can also periodically update the machine learning classification model based on indicators of stress experienced by the device wearer and, thus, make the detection of stress personalized for the individual.

In various embodiments, an ear-wearable stress monitoring system is included having a control circuit, a microphone, a sensor package, and an electroacoustic transducer that can be configured for placement about or within an ear canal of the device wearer. The ear-wearable stress monitoring system can be configured to evaluate data from at least one of the microphone and the sensor package and classify a device wearer's stress level using a machine learning classification model. The system can further provide information to the device wearer relating to the classified stress level in a discrete manner so that the device wearer can receive information useful to them while keeping the information confidential with respect to others who may be near the device wearer.

In an embodiment, an ear-wearable stress monitoring system is included having a control circuit, a microphone, an electroacoustic transducer, and a sensor package. The ear-wearable stress monitoring system can be configured to detect an occurrence of stress of the device wearer exceeding a threshold value and evaluate data from at least one of the microphone and the sensor package over a lookback period to detect a trigger of the stress. In this way, the system/device can identify stressors (or causes of stress) that are specifically applicable for the device wearer.

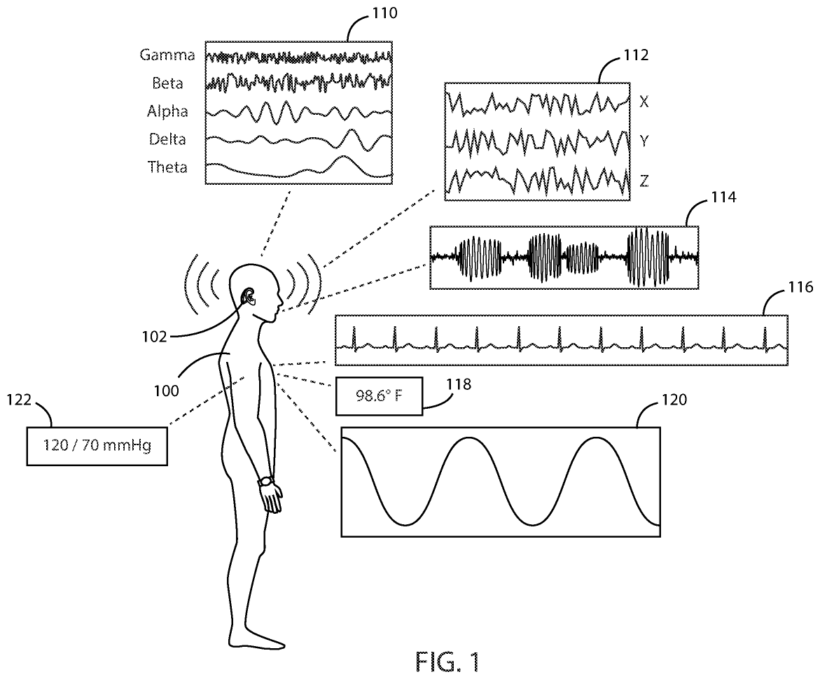

In various embodiments, the system and/or device can detect many different possible markers of stress. Referring now to FIG. 1, a schematic view is shown of some physiological indicators of stress that can be detected in accordance with embodiments herein. In specific, FIG. 1 illustrates a device wearer 100 with an ear-wearable device 102. The ear-wearable device 102 can be used to directly sense and/or receive information regarding various sensed parameters relating to stress. For example, the ear-wearable device 102 can include a sensor package (described in greater detail below) that can sense parameters including, but not limited to, electroencephalogram (EEG) data 110, accelerometer data 112 and/or gyroscope data from a motion sensor, the device wearer's voice 114, electrocardiogram (ECG) data 116 which can be used to determine heart rate and heart rate variability amongst other things, temperature data 118, respiratory data 120, and blood pressure data 122. It will be appreciated, however, that the sensed parameters shown in FIG. 1 are only examples and that various other parameters and data are also contemplated herein. For example, sympathetic nervous stimulation may impact (typically reduce) urination. As such, urination events (which can be done using microphone data, inputs from the device wearer or a third party, etc.) can also be used as data herein for the evaluation of stress and/or anxiety. As another example, electromyography (EMG) data can also be used herein as muscular tonus is often increased while an individual is experiencing stress. EMG data can be evaluated to sense, for example, auricular muscle contraction or tone and/or jaw muscle contraction or tone which can be used as data herein to detect stress. In some cases, eye movement and/or pupil dilation can be evaluated herein to detect stress. Aspects of detecting eye movement and pupil dilation as described in U.S. Publ. Pat. Appl. No. 2020/0143703, the content of which is herein incorporated by reference.

In various embodiments, indicators of stress experienced by the device wearer 100 can be derived from data produced by at least one of the microphone and the sensor package. In various embodiments, the sensor package can specifically include at least one including at least one of a heart rate sensor, a heart rate variability sensor, an electrocardiogram (ECG) sensor, a blood oxygen sensor, a blood pressure sensor, a skin conductance sensor, a photoplethysmography (PPG) sensor, a temperature sensor (to measure one or more of core body temperature, skin temperature, etc.), a motion sensor, an electroencephalograph (EEG) sensor, and a respiratory sensor.

An acute response to stress can include, for example, one or more of (with respect to a baseline value) an increased heart rate, decreased heart rate variability, increased respiration rate, increased blood pressure, increased core temperature, changes in motion data such as that consistent with displacement behaviors and other nervous habits, and changes in the pitch (such as an increased pitch) and/or volume (such as an increased volume) of the device wearer's voice, amongst others. Baseline values for all of these parameters can be determined by the system/device over time as the ear-wearable device is being worn. Baseline values can be important to establish as these values are typically unique to individuals. For example, resting heart rates vary substantially across individuals, as well as the quantum of heart rate increase in response to stress. In various embodiments, the device can enter a baseline establishment mode where for a period of time spanning hours, days, weeks, or even months all of these types of data are tracked and then subjected to statistical operations in order to set baseline values.

Changes over baseline values deemed to have significance can be set as a default value, can be programmed in by the device wearer or a third party, or can be related to a statistical measure of baseline values such as in units of standard deviation. In various embodiments, changes over a baseline value of greater than or equal to 5, 15, 25, 35, 45, 55, 65, 75, 85, 95, 100, 150, or 200 percent, or an amount falling within a range between any of the foregoing, can be deemed to be a marker of an episode of stress experienced by the device wearer. In some embodiments, the combination of measured parameters reflecting baseline or a non-stressed state can be compared with a current combination of measured parameters using machine learning approaches or other statistical approaches to determine whether the current state substantially matches the baseline state (e.g., the device wearer is not currently stressed) or is different than the baseline state (e.g., the device wearer is currently experiencing stress). In some embodiments, determinations can, in some cases, be binary (not stressed vs. stressed). In other embodiments, determinations can be non-binary reflecting degrees of stress being experienced by the device wearer. In various embodiments herein, data regarding measured parameters and other data can be used to classify a stress level of a device wearer using a machine learning classification model as described in greater detail below.

Depending on the individual, some of these acute physiological changes in response to stress may be more prominent in some individuals versus others. As such, as described further below, systems and devices herein can adapt to an individual and thus more accurately detect the onset and/or changes in stress. Displacement behaviors, as one specific example, may be particularly subject to differences across individuals. As such, in various embodiments, the ear-wearable stress monitoring system can be configured to detect displacement behaviors and correlate classified stress levels with detected displacement behaviors. For example, if for a given individual stress is frequently accompanied by a specific displacement behavior (such as one that is recognized by a particular pattern of a motion sensor or microphone such as shaking of their legs, arms, making certain noises, tapping, etc.) as revealed by a correlation between the two, then in future scenarios the detection of the displacement behavior can be effectively weighted more heavily, such that the system is more likely to categorize a level of stress as being significant if the specific displacement behavior is also present. Correlations described herein can be derived using standard statistical technique that can show whether and how strongly pairs of variables and/or pairs of groups of variables are related.

In some embodiments, the ear-wearable stress monitoring system (described further below) can be configured to distinguish between acute and chronic stress of the device wearer 100. Markers of acute stress can include parameters such as increased heart rate, which is an almost-instantaneous sympathetic nervous response to stress. Markers of chronic stress can overlap with markers of acute stress, but can also include markers that are largely distinct including trouble sleeping which can manifest, for example, as unusual movement detected by motion sensors during sleeping hours, increased background levels of displacement behaviors, fatigue which can manifest, for example, as decreased physical activity levels during waking hours, and the like. Embodiments herein can be configured to detect these markers of chronic stress.

In various embodiments, the time scale of certain markers can be used to delineate between acute stress and chronic stress. For example, increased heart rate that rises transitorily then falls back to a normal level (unaccompanied by motion data consistent with physical exertion) is more likely to indicate acute stress. In contrast, a chronic elevation in heart rate is more likely to reflect chronic stress. Embodiments herein can be configured to calculate the time scale of changes in measured parameters that can be used to distinguish between acute and chronic stress. In some cases, those changes with a time scale crossing a threshold value can be deemed to be markers of chronic stress. The threshold value can be minutes, hours, days, weeks or months. Therefore, the system/device can delineate between that which is an acute stress episode which is experienced by the device wearer and changes in levels of chronic stress experienced by the device wearer.

In some examples, the specificity or sensitivity (or both) of detection operations herein can be improved by detection of an event or circumstance that is not necessarily related to stress or anxiety, but nonetheless generates signals that could be confused with those reflecting stress or anxiety. For example, a device or system herein can determine an activity classification, such as running, sitting, or walking, and the activity classification can be considered as an input to a stress or anxiety evaluation scheme. In an example, physiological changes (e.g., increased blood pressure, heart rate, or respiration rate) in a person who is sitting or walking slowly may correlate with stress, but if the person is actively exercising (e.g., running), such changes may be caused by physical exertion—not stress or anxiety. Other data, such as geolocation data, may also be used in this manner. For example, the fact that a person is walking uphill (e.g., determined from geolocation) can be considered as a factor to evaluate the likelihood that a physiologic signals are related to emotional stress or anxiety as opposed to physical exertion.

In some examples, the cause of a physiological signals correlated with stress may be inferred from data, such as environmental classification information or geolocation. For example, the fact that a person is in a particular environment (e.g., near a roaring crowd or busy street) or near a particular location (e.g., near a roller coaster, haunted house, movie theater or other stress-inducing local) can be used by the system to infer whether physiological signals are a response to such an environment (e.g., as opposed to internal or emotional stress.)

In some embodiments, the system can record and/or analyze how the device wearer interfaces with the ear-wearable device during an episode of stress. For example, the device wearer may adjust operational parameters such as a gain value (controlling volume of sound provided by the device) up or down, may adjust ambient sound attenuation or cancellation settings, may make tinnitus masker stimulus volume adjustments, may turn tinnitus stimulus on or off, may change a tinnitus masker type, or any number of other changes. By recording such changes, the device can be configured to automatically make the same changes (or at least some of the same changes) the next time an episode of stress is detected so that the ear-wearable device automatically adjusts itself as desired by the device wearer during an episode of stress. In some embodiments, the device or system can query the user regarding the automatic adjustments to confirm their desirability. For example, in the case of automatically changing a tinnitus stimulus the device or system could pose a question to the device wearer such as “does this sound provide more relief?” Depending on the response from the device wearer, the change can either be kept or rolled back.

One aspect of monitoring stress relates to detecting stress experienced by a device wearer as described above and further below. However, in order to determine the most effective way to mitigate stress and/or suggest various interventions, it is important to understand the causes of stress for a particular individual. In various embodiments, the system and/or device can detect many different possible stressors or causes of stress and, in some embodiments, empirically determine the impact of those stressors on the individual so that the relationship between possible stressors and stress episodes for a given individual can be elucidated. In this way, the device can customize possible interventions to be most effective for a given individual.

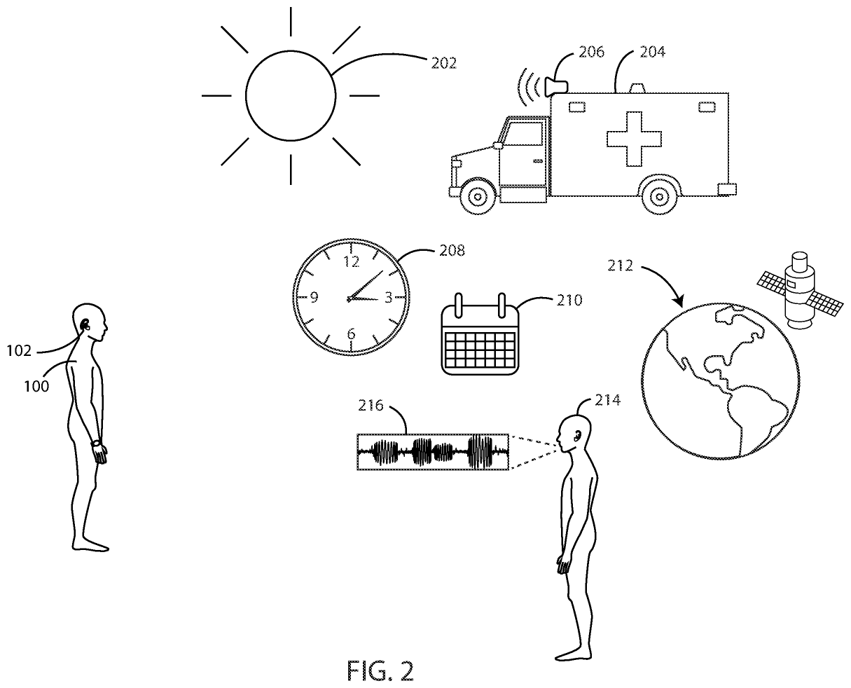

Referring now to FIG. 2, a schematic view is shown of possible stressors and other items related to stress in accordance with various embodiments herein. In specific, FIG. 2 depicts a device wearer 100 with an ear-wearable device 102 that can be part of an ear-wearable stress monitoring system. Possible stressors can include environmental conditions 202 of all types including, but not limited to, weather patterns, temperature, humidity, barometric pressure, and the like. In various embodiments, the ear-wearable stress monitoring system can be configured to characterize the stress causing potential of environmental conditions 202 by observing/detecting markers of stress occurring simultaneously and/or subsequent to specific environmental conditions 202. Similarly, the stress relieving potential of environmental conditions 202 can be determined for the individual by observing/detecting changes in stress (such as decreases) experienced by an individual in response to simultaneous or preceding environmental conditions 202. Such information can be used by the system/device in order to suggest effective stress interventions for an individual. If, for example, a sunny day is observed to have a stress reducing impact on a given individual, then when an intervention is needed a suggestion to “go outside” can be issued by the system when it detects that the day is sunny (directly through sensors or indirectly through the use of a weather API or the like). Such suggestions can be consistent with operations of providing suggestions or interventions as describe in greater detail below.

Possible stressors can also include things such as objects making noise within the environment of the device wearer (ambient sounds) including, for example, vehicles 204 creating vehicle noise 206 which can be, without limitation, passenger vehicles, commercial vehicles, trucks, trains, heavy equipment, airplanes, and the like. Sources of ambient sounds can also include people, animals, other machinery, and the like. Not all sounds are the same in terms of their stress causing potential. For example, while particularly loud and sudden sounds (such as a gunshot) may generate substantial acute stress, sounds that are loud but more constant (such as noises associated with riding in a vehicle) may not generate nearly as much acute stress. However, such loud and constant noise may still result in adverse chronic stress. In various embodiments, the ear-wearable stress monitoring system can be configured to characterize the stress causing potential of ambient sound by observing/detecting markers of stress occurring simultaneously and/or subsequent to specific ambient sounds.

In some embodiments, time 208 (such as time of day) can also be an item relevant for considering with respect to stress as stress levels and/or susceptibility to being stressed can follow a diurnal pattern. For example, the steroid hormone cortisol is one of two main peripheral secretory products of the hypothalamic-pituitary-adrenal stress-neuroendocrine axis and typically follows a strong diurnal rhythm with levels are high on waking, surge in the 30-40 minutes after waking, drop rapidly in subsequent few hours after the awakening surge and then drop more slowly until reaching a nadir around bedtime. The levels of cortisol in a given individual may impact their reactions to possible stressors. Thus, in various embodiments herein, the system/device can utilize information regarding the time of day in order to characterize an individual's stress levels as well as the individual's susceptibility to various possible stressors.

The day of week or month, such as represented by calendar 210, can also be an item relevant for consideration/evaluation with respect to stress detection and/or monitoring as it may relate to personal or work schedules, habits, activities, physiological cycles, and the like. For example, individuals may characteristically be more stressed or susceptible to stress on Mondays if, for example, that represents the beginning of their working week. Similarly, hormonal changes associated with menstrual cycles may impact stress levels of some individuals. In various embodiments, the ear-wearable stress monitoring system 700 can be configured to characterize the impact of the day of the week on potential stressors by observing/detecting patterns of potential stressors and simultaneous or subsequent markers of stress as a function of the day of the week (or other time measure) when the potential stressor and stress response occurs.

The geolocation 212 of the individual can also be an item relevant for consideration/evaluation with respect to stress detection and/or monitoring. In various embodiments, the ear-wearable stress and anxiety monitoring system can be configured to characterize the stress causing potential of a specific geolocation 212 by observing/detecting markers of stress occurring simultaneously and/or subsequent to the presence of the device wearer at a specific geolocation 212. Geolocation 212 can be determined via a geolocation circuit of the system or device.

Whether or not the device wearer has gotten sufficient sleep can greatly impact their physiological response to various possible stressors. Most individuals will generally experience more stress from a given possible stressor if they are lacking sufficient sleep than if they are well rested. As such, in various embodiments herein sleep can be evaluated as a piece of data along with other factors as described. For example, in various embodiments, the system and/or device can monitor and evaluate microphone and/or sensor data herein to detect patterns consistent with sleeping and record the same for evaluation along with other data herein when determining the presence of stress and/or evaluating whether or not specific possible stressors serve as stressors for the given device wearer. In some embodiments, the system and/or device can receive information regarding sleep from the device wearer as an input, from a third party as an input, from another device such as a sleep monitoring device, or from an electronic medical record system or the like.

Possible stressors can also include various third parties 214. FIG. 2 depicts a third party 214 along with third party communication 216. The third party 214 could be a family member, a care provider, a coworker, a manager, or the like. In various embodiments, the ear-wearable stress and anxiety monitoring system can be configured to evaluate data from the microphone or other sensors indicating the presence of an individual serving as a possible stressor or trigger of stress. In various embodiments, the ear-wearable stress monitoring system can specifically be configured to evaluate data from the microphone to identify a voice of an individual serving as a stressor or trigger of stress. It will be appreciated, however, that the system can also identify specific third parties through techniques other than analyzing voices. For example, electronic devices carried by third parties (devices personal to them such as smart phones or other personal electronics) can send advertising packets or other wireless data communications that can be received by the system or device(s) herein and can include data therein that can be used to identify a specific device and, by proxy, a specific third party. In various embodiments, the ear-wearable stress monitoring system can be configured to characterize the stress causing potential of the presence of a specific third party 214 by observing/detecting markers of stress occurring simultaneously and/or subsequent to the presence of the specific third party 214.

Stressors can be highly variable amongst individuals. As such, in various embodiments, the ear-wearable stress monitoring system can be configured to learn what possible stressors generate stress for an individual by correlating possible detected stressors with subsequent classified stress levels of the device wearer 100. In various embodiments, the ear-wearable stress monitoring system can be configured to weight certain possible detected stressors in the classification model more heavily based on the correlation other statistical measures. In some embodiments, this weighting can be applied explicitly by the system/device. In some embodiments, this weighting can be applied through the generation of a machine learning model which includes such information as inputs.

The identity of the individual generating speech or other sounds can be tremendously important in determining whether specific speech or sounds are a marker of stress experienced by the device wearer or a possible stressor that may lead to stress for the device wearer. For example, in various embodiments, the voice of the third party can be treated as a possible detected stressor. As such, in various embodiments, wherein data from the microphone can be processed to distinguish a voice of the device wearer from a voice of a third party (exemplary aspects of own-voice detection are described in greater detail below).

Distinguishing between speech or sounds associated with the device wearer and speech or sounds associated with a third party can be performed in various ways. In some embodiments, this can be performed through signal analysis of the signals generated from the microphone(s). For example, in some embodiments, this can be done by filtering out frequencies of sound that are not associated with speech of the device-wearer. In some embodiments, such as where there are two or more microphones (on the same ear-wearable device or on different ear-wearable devices) this can be done through spatial localization of the origin of the speech or other sounds and filtering out, spectrally subtracting, or otherwise discarding sounds that do not have an origin within the device wearer. In some embodiments, such as where there are two or more ear-worn devices, own-voice detection can be performed and/or enhanced through correlation or matching of intensity levels and or timing.

In some cases, the system can include a bone conduction microphone to preferentially pick up the voice of the device wearer. In some cases, the system can include a directional microphone that is configured to preferentially pick up the voice of the device wearer. In some cases, the system can include an intracanal microphone (a microphone configured to be disposed within the ear-canal of the device wearer) to preferentially pick up the voice of the device wearer. In some cases, the system can include a motion sensor (e.g., an accelerometer configured to be on or about the head of the wearer) to preferentially pick up skull vibrations associated with the vocal productions of the device wearer.

In some cases, an adaptive filtering approach can be used. By way of example, a desired signal for an adaptive filter can be taken from a first microphone and the input signal to the adaptive filter is taken from the second microphone. If the hearing aid wearer is talking, the adaptive filter models the relative transfer function between the microphones. Own-voice detection can be performed by comparing the power of an error signal produced by the adaptive filter to the power of the signal from the standard microphone and/or looking at the peak strength in the impulse response of the filter. The amplitude of the impulse response should be in a certain range to be valid for the own voice. If the user's own voice is present, the power of the error signal will be much less than the power of the signal from the standard microphone, and the impulse response has a strong peak with an amplitude above a threshold. In the presence of the user's own voice, the largest coefficient of the adaptive filter is expected to be within a particular range. Sound from other noise sources results in a smaller difference between the power of the error signal and the power of the signal from the standard microphone, and a small impulse response of the filter with no distinctive peak. Further aspects of this approach are described in U.S. Pat. No. 9,219,964, the content of which is herein incorporated by reference.

In another approach, the system uses a set of signals from a number of microphones. For example, a first microphone can produce a first output signal A from a filter and a second microphone can produce a second output signal B from a filter. The apparatus includes a first directional filter adapted to receive the first output signal A and produce a first directional output signal. A digital signal processor is adapted to receive signals representative of the sounds from the user's mouth from at least one or more of the first and second microphones and to detect at least an average fundamental frequency of voice (pitch output) F₀. A voice detection circuit is adapted to receive the second output signal B and the pitch output F₀ and to produce an own voice detection trigger T. The apparatus further includes a mismatch filter adapted to receive and process the second output signal B, the own voice detection trigger T, and an error signal E, where the error signal E is a difference between the first output signal A and an output O of the mismatch filter. A second directional filter is adapted to receive the matched output O and produce a second directional output signal. A first summing circuit is adapted to receive the first directional output signal and the second directional output signal and to provide a summed directional output signal (D). In use, at least the first microphone and the second microphone are in relatively constant spatial position with respect to the user's mouth, according to various embodiments. Further aspects of this approach are described in U.S. Pat. No. 9,210,518, the content of which is herein incorporated by reference.

In various embodiments herein, a device or system can specifically include an inward-facing microphone (e.g., facing the ear canal, or facing tissue, as opposed to facing the ambient environment.) A sound signal captured by the inward-facing microphone can be used to determine physiological information, such as that relating to a physiological response to stress or anxiety. For example, a signal from an inward-facing microphone may be used to determine heart rate, respiration, or both, e.g., from sounds transferred through the body. In some examples, a measure of blood pressure may be determined, e.g., based on an amplitude of a detected physiologic sound (e.g., louder sound correlates with higher blood pressure.)

Knowledge of meals can be an important consideration when detecting, monitoring, and/or predicting stress. In general, consuming a meal has the effect of reducing stress of an individual. However, this may not be consistently true for all individuals depending on their metabolism and other factors. Similarly, stress may rise along with the amount of time that has passed since a previous meal as the individual becomes hungry. As such, in various embodiments, the ear-wearable stress monitoring system can be configured to detect meals and correlate classified stress levels with detected meals and/or the amount of time that has passed since a previous meal. Similar to meals, knowledge of medication events (e.g., when a device wearer takes or otherwise receives a medication) can be an important consideration when detecting, monitoring, and/or predicting stress. In some scenarios, the drinking of fluids can be similar to food intake with respect to its impact on stress. In some cases, excessive food intake and/or insufficient hydration can result in increased stress and/or anxiety.

In various embodiments, the ear-wearable stress monitoring system can be configured to detect meals and/or fluid intake of the device wearer and cross-reference classified stress levels against detected meals and/or fluid intake events. In some embodiments, meals or fluid intake events can be identified based on identifying or matching characteristic patterns in the data from a microphone and/or other sensors such as motions sensors herein. For example, a “positive” pattern for sensor data associated with a meal or a fluid intake event can be stored by the system and current data can be periodically matched against such a pattern. If a match exceeding a threshold value is found, then a meal or a fluid intake event can be deemed to have taken place. Further details regarding meal and fluid intake detection are provided in U.S. Pat. Appl. No. 63/058,936, titled “Ear-Worn Devices with Oropharyngeal Event Detection”, the contents of which are herein incorporated by reference in its entirety. Further details regarding medication event (such as taking or receiving a medication) detection are provided in U.S. Publ. Pat. Appl. No. 2020/0268315, titled “System and Method for Managing Pharmacological Therapeutics Including a Health Monitoring Device”, the contents of which are herein incorporated by reference in its entirety.

In various embodiments, the ear-wearable stress monitoring system (described further below) can be configured to predict a future occurrence of stress of the device wearer 100. Such predictions can be based upon various factors including, but not limited to, those potential stressors describe above and/or at least one of current inputs from the microphone and/or sensor package, information from a calendar of the device wearer, and a geographic location of the device wearer. Predictions can be particularly useful because predictions can allow suggestions or interventions to be issued by the system/device before the device wearer is currently in the midst of an episode of stress. Further aspects of predictions are described in greater detail below.

Ear-wearable devices herein, including hearing aids and hearables (e.g., wearable earphones), can include an enclosure, such as a housing or shell, within which internal components are disposed. Components of an ear-wearable device herein can include a control circuit, digital signal processor (DSP), memory (such as non-volatile memory), power management circuitry, a data communications bus, one or more communication devices (e.g., a radio, a near-field magnetic induction device), one or more antennas, one or more microphones, a receiver/speaker, a telecoil, and various sensors as described in greater detail below. More advanced ear-wearable devices can incorporate a long-range communication device, such as a BLUETOOTH® transceiver or other type of radio frequency (RF) transceiver.

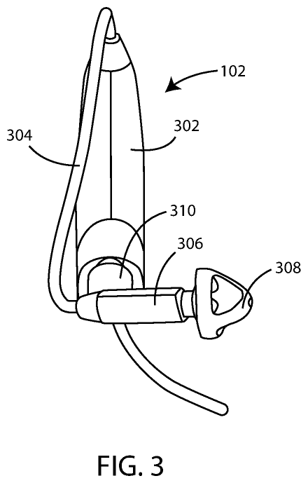

Referring now to FIG. 3, a schematic view of an ear-wearable device 102 is shown in accordance with various embodiments herein. The ear-wearable device 102 can include a hearing device housing 302. The hearing device housing 302 can define a battery compartment 310 into which a battery can be disposed to provide power to the device. The ear-wearable device 320 can also include a receiver 306 adjacent to an earbud 308. The receiver 306 an include a component that converts electrical impulses into sound, such as an electroacoustic transducer, speaker, or loudspeaker. Such components can be used to generate an audible stimulus in various embodiments herein. A cable 304 or connecting wire can include one or more electrical conductors and provide electrical communication between components inside of the hearing device housing 302 and components inside of the receiver 306.

The ear-wearable device 102 shown in FIG. 3 is a receiver-in-canal type device and thus the receiver is designed to be placed within the ear canal. However, it will be appreciated that many different form factors for ear-wearable devices are contemplated herein. As such, ear-wearable devices herein can include, but are not limited to, behind-the-ear (BTE), in-the ear (ITE), in-the-canal (ITC), invisible-in-canal (ITC), receiver-in-canal (RIC), receiver in-the-ear (RITE), completely-in-the-canal (CIC) type hearing assistance devices, a personal sound amplifier, a cochlear implant, a bone-anchored or otherwise osseo-integrated hearing device, or the like.

While FIG. 3 shows a single ear-wearable device, it will be appreciated that in various examples, a pair of ear-wearable devices can be included and can work as a system, e.g., an individual may wear a first device on one ear, and a second device on the other ear. In some examples, the same type(s) of sensor(s) may be present in each device, allowing for comparison of left and right data for data verification (e.g., increase sensitivity and specificity through redundancy), or differentiation based on physiologic location (e.g., physiologic signal may be different in one location from the other location.)

Ear-wearable devices of the present disclosure can incorporate an antenna arrangement coupled to a high-frequency radio, such as a 2.4 GHz radio. The radio can conform to an IEEE 802.11 (e.g., WIFI®)) or BLUETOOTH® (e.g., BLE, BLUETOOTH® 4. 2 or 5.0) specification, for example. It is understood that ear-wearable devices of the present disclosure can employ other radios, such as a 900 MHz radio. Ear-wearable devices of the present disclosure can be configured to receive streaming audio (e.g., digital audio data or files) from an electronic or digital source. Representative electronic/digital sources (also referred to herein as accessory devices) include an assistive listening system, a TV streamer, a remote microphone device, a radio, a smartphone, a cell phone/entertainment device (CPED), a programming device, or other electronic device that serves as a source of digital audio data or files.

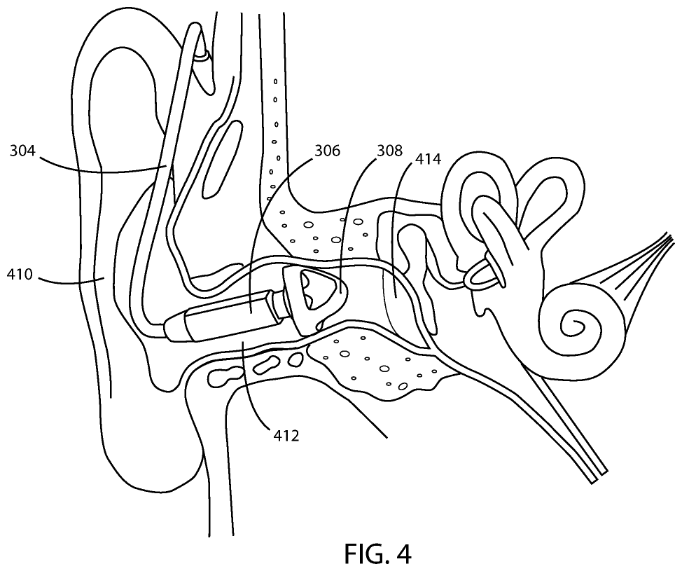

As mentioned above, the ear-wearable device 102 can be a receiver-in-canal type device and thus the receiver is designed to be placed within the ear canal. Referring now to FIG. 4, a schematic view is shown of an ear-wearable device disposed within the ear of a subject in accordance with various embodiments herein. In this view, the receiver 306 and the earbud 308 are both within the ear canal 412, but do not directly contact the tympanic membrane 414. The hearing device housing is mostly obscured in this view behind the pinna 410, but it can be seen that the cable 304 passes over the top of the pinna 410 and down to the entrance to the ear canal 412.

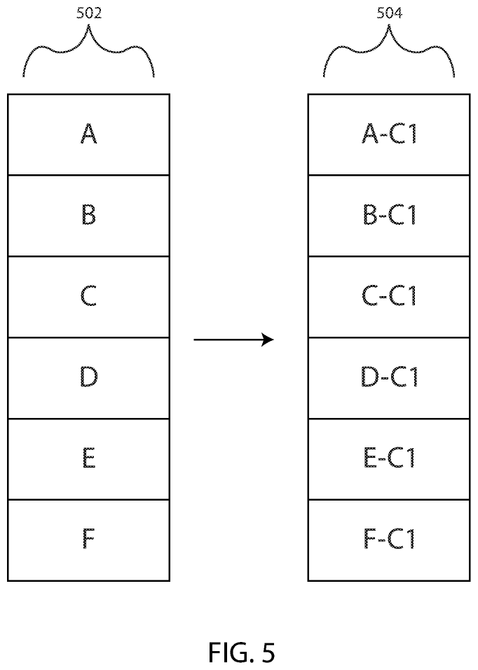

Referring now to FIG. 5, a schematic view is shown of classification models in accordance with various embodiments herein. An ear-wearable stress monitoring system can include and/or utilize a first or default machine learning classification model 502. In this example, the ear-wearable stress monitoring system also includes a customized classification model 504, wherein the customized classification model 504 is specific for the device wearer and is created over time as the device wearer utilizes the ear-wearable stress monitoring system.

The ear-wearable stress monitoring system can utilize data from any of the sensors described herein and/or any of the sources of data described herein (e.g., indicators of stress) in a machine learning approach to categorize a current level of stress being experienced by the device wearer. For example, the ear-wearable stress monitoring system can be configured to evaluate data from at least one of the microphone and the sensor package and classify a stress level of a device wearer 100 using a machine learning classification model 502 and periodically update the machine learning classification model to generate a second or customized machine learning classification model 502 based on indicators of stress experienced by the device wearer.

In some embodiments, the initial or default machine learning classification model can be generated using sets of data gathered from individuals numbering in the hundreds, or thousands, or more. The initial or default machine learning classification model can be generated using supervised or unsupervised machine learning approaches.

In various embodiments, the ear-wearable stress monitoring system (described further below) can be configured to weight certain possible detected stressors in the machine learning classification model 502 more heavily based on the correlation.

In some embodiments, the system can more accurately sense stress if a model is used that is specific for individuals sharing some characteristics with the individual wearing the ear-wearable device. For example, a model can be used wherein the model is specific for individuals of a certain gender falling within a specific age range. Many other factors can be used including, for example, health status, weight, medical history, and the like.

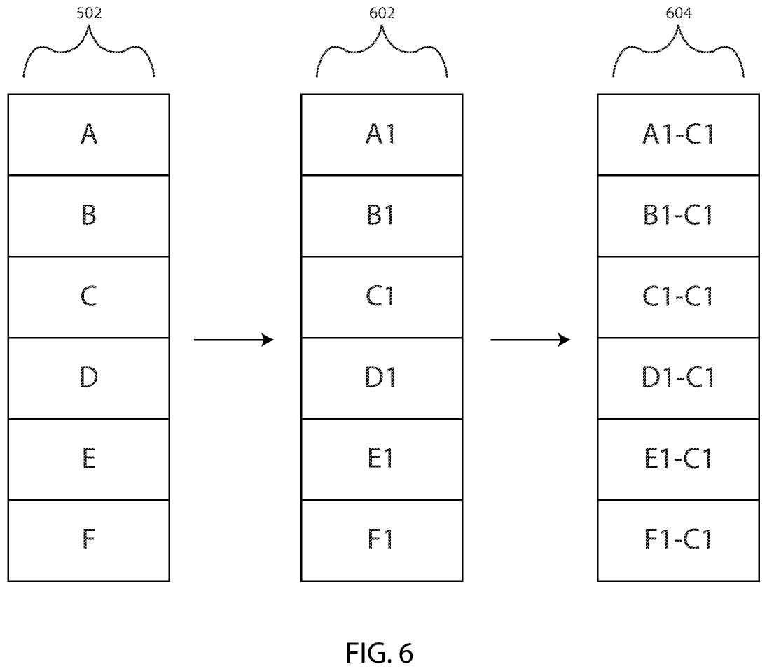

Referring now to FIG. 6, a schematic view of classification models is shown in accordance with various embodiments herein. In this embodiment, if the system can determine or be provided with certain characteristics of the individual wearing the ear-wearable device, instead of starting with a default machine learning classification model 502, the ear-wearable stress monitoring system can start with a characteristic or demographic specific classification model 602. Using that model as a starting point, the ear-wearable stress monitoring system can further modify/update the model based on data while the individual is wearing the device to generate a customized model 604 for later use. In some embodiments, the customized model 604 can be updated indefinitely.

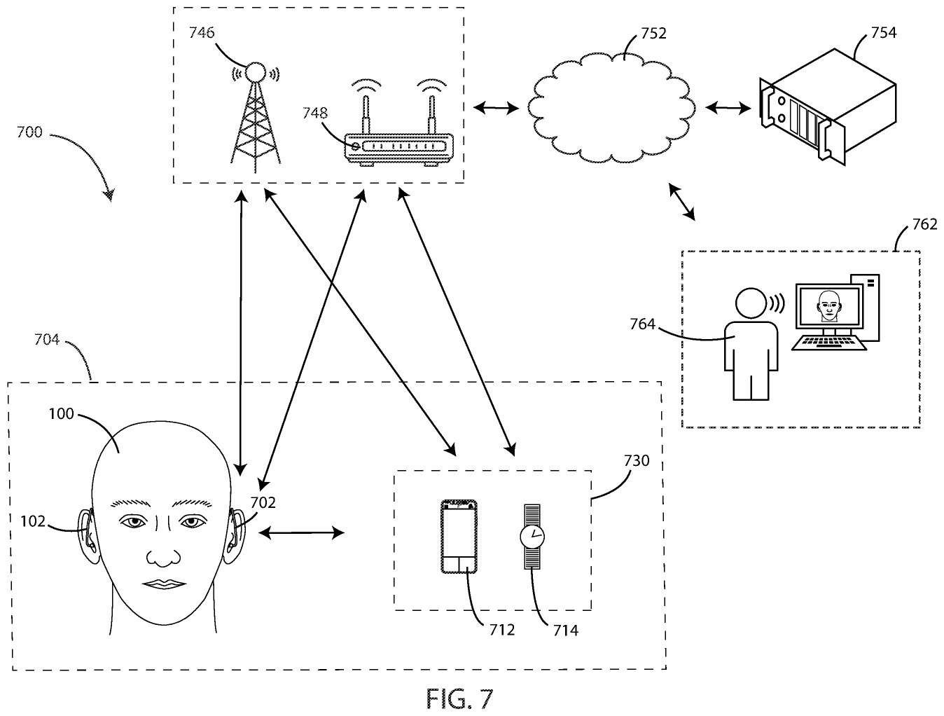

Referring now to FIG. 7, a schematic view of an ear-wearable stress monitoring system 700 is shown in accordance with various embodiments herein. FIG. 7 shows a device wearer 100 with an ear-wearable device 102 and a second ear-wearable device 702. The device wearer 100 is at a first location or patient location 704. The system can include and/or can interface with other devices 730 at the first location 704. The other devices 730 in this example can include an accessory device 712, which could be a smart phone or similar mobile communication/computing device in some embodiments. The other devices 730 in this example can also include a wearable device 714, which could be an external wearable device 714 such as a smart watch or the like.

FIG. 7 also shows communication equipment including a cell tower 746 and a network router 748. FIG. 7 also schematically depicts the cloud 752 or similar data communication network. FIG. 7 also depicts a cloud computing resource 754. The communication equipment can provide data communication capabilities between the ear-wearable devices 102, 702 and other components of the system and/or components such as the cloud 752 and cloud resources such as a cloud computing resource 754. In some embodiments, the cloud 752 and/or resources thereof can host an electronic medical records system. In some embodiments, the cloud 752 can provide a link to an electronic medical records system.

FIG. 7 also shows a remote location 762. The remote location 762 can be the site of a third party 764, which can be a clinician, care provider, loved one, or the like. The third party 764 can receive reports regarding the identified stress levels and/or stressors of the device wearer. In some embodiments, the third party 764 can provide instructions for the device wearer regarding actions to take, such as actions to reduce or alleviate their stress.

In various embodiments, the ear-wearable stress monitoring system 700 can be configured to send information regarding classified stress levels to an electronic medical record system. In various embodiments, the ear-wearable stress monitoring system 700 can be configured to send information regarding classified stress levels to a third party 764. In some embodiments, the ear-wearable stress monitoring system 700 can be configured to receive information regarding stress, stressors, or classified stress levels (such as stress testing information that may have been derived/performed in-clinic) as relevant to the individual through an electronic medical record system. Such received information can be used alongside data from microphones and other sensors herein and/or incorporated into machine learning classification models used herein.

In various embodiments, ear-wearable stress monitoring systems can be configured so that indicators of stress experienced by the device wearer are derived from inputs provided by a device wearer. Such inputs can be direct inputs (e.g., an input that is directly related to stress or anxiety) or indirect inputs (e.g., an input that relates to or otherwise indicates a condition of stress but is not directed specifically to stress). For example, in various embodiments, the ear-wearable stress monitoring system can be configured so that a device wearer input in the form of a “tap” of the device can signal that the device wearer is experiencing stress. In some embodiments, the ear-wearable stress monitoring system can be configured to generate a query for the device wearer and the device wearer input can be in the form of a response to the query. As an example of an indirect input, because tinnitus may worsen when the device wearer is experiencing stress or anxiety the device wearer may make adjustments to tinnitus management settings on the system or device and this can be taken by the system as an indicator that they are experiencing stress or anxiety.

However, it will be appreciated that in various embodiments, indicators of stress experienced by the device wearer can be received by the ear-wearable stress monitoring system from an external source.

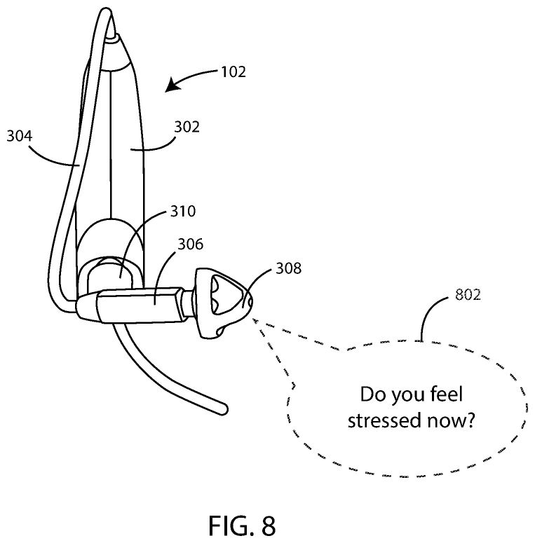

Referring now to FIG. 8, a schematic view of an ear-wearable device 102 is shown in accordance with various embodiments herein. The ear-wearable device 102 can be part of a stress monitoring system. The ear-wearable device 102 can include a housing 302, a cable 304, a receiver 306, an earbud 308, and a battery compartment 310. The ear-wearable stress monitoring system can be configured to generate and issue a wearer query 802. In some embodiments, the query 802 can be issued audibly by the ear-wearable device 102. However, by virtue of an electroacoustic transducer (or speaker) of the ear-wearable device 102 be positioned within or adjacent to the ear canal of the device wearer, the query 802 can be provided at a volume that can only be heard by the device wearer and thus discretely. The device wearer can respond to the query 802 in various ways. For example, in some embodiments, the device wearer can respond by way of a tap. In some embodiments, the device wearer can respond by way of a spoken answer that can be received by way of a microphone of the ear-wearable device. In some embodiments, the device wearer can respond by way of a specific gesture that can be identified by analyzing data from a motion sensor herein such as a head nod, head shake, or other head or body gesture.

In some embodiments herein, queries to the device wearer can be generated and/or issued to the device wearer using a different device. For example, in some embodiments, an accessory device can be used to present a query to the device wearer.

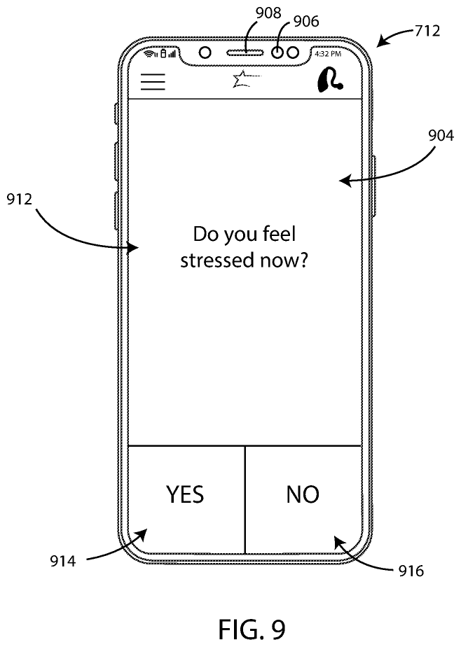

Referring now to FIG. 9, a schematic view of an accessory device 712 is shown in accordance with various embodiments herein. The accessory device 712 includes a display screen 904. The accessory device 712 also includes a camera 906 and a speaker 908. The accessory device 712 can generate and/or present an accessory query 912. In order to receive input from the device wearer, the accessory device 712 can also include, for example, a first user input object 914 and a second user input object 916. Thus, in various embodiments, the indicators of stress and/or confirmation of stress experienced by the device wearer can be derived from inputs provided by the device wearer and/or can be received by the ear-wearable stress monitoring system from an external source.

In various embodiments herein, the ear-wearable stress monitoring system can be configured to provide various pieces of information to the device wearer relating to the classified stress level and/or relating to suggestions for actions to take. In many embodiments herein, the ear-wearable stress monitoring system can be configured to provide information to the device wearer relating to the classified stress level in a discrete manner. For example, in various embodiments, the ear-wearable stress monitoring system can provide information relating to the classified stress level through an electroacoustic transducer at volume that only the device wearer can hear.

The information provided to the device wearer relating to the classified stress level can take many forms. In some embodiments, the information can comprise verbal information. In some embodiments, the information provided to the device wearer relating the classified stress level can include non-verbal sound(s). By way of example, in various embodiments, non-verbal sounds provided by the ear-wearable device can include music. In various embodiments, the information relating to the classified stress level includes a sound preselected by the device wearer.

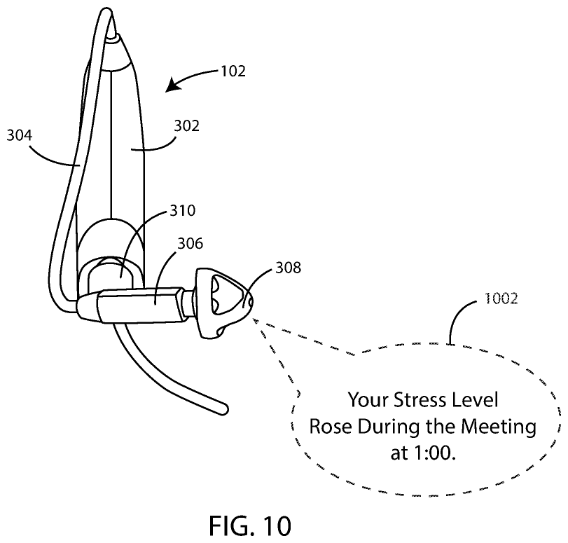

Referring now to FIG. 10, a schematic view of an ear-wearable device 102 is shown in accordance with various embodiments herein. The ear-wearable device 102 can be part of an ear-wearable stress monitoring system. The ear-wearable device 102 includes a housing 302, a cable 304, a receiver 306, an earbud 308, and a battery compartment 310. The ear-wearable stress monitoring system can be configured to provide information 1002 to the device wearer, such as in a discrete manner. In various embodiments, the information relating to the classified stress level can be provided through an electroacoustic transducer that can be part of the receiver 306.

In various embodiments, the ear-wearable stress monitoring system can be configured to provide instructions regarding recommended interventions or mitigating actions to the device wearer. As such, in various embodiments, information relating to the classified stress level provided by the ear-wearable stress monitoring system includes instructions for an action for the device wearer to take to reduce the stress level of the device wearer. In various embodiments, the instructions for an action to take specifically include breathing instructions. Aspects of breathing exercises and instructions can be found in U.S. Publ. Appl. No. 2020/0008708, the content of which is herein incorporated by reference. In various embodiments, the instructions for an action to take specifically include a suggestion to meditate. In various embodiments, the instructions for an action to take can specifically include a suggestion to consume a food item. In various embodiments, the instructions for an action to take can specifically include a suggestion to take a medication.

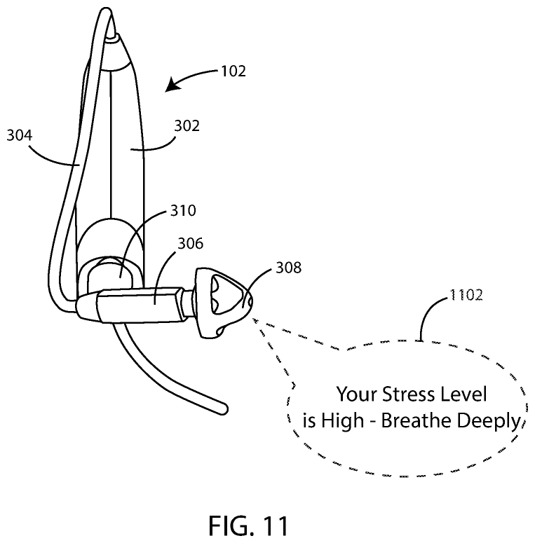

Referring now to FIG. 11, a schematic view of an ear-wearable device 102 is shown in accordance with various embodiments herein. The ear-wearable device 102 can be part of an ear-wearable stress monitoring system. The ear-wearable device 102 includes a housing 302, cable 304, receiver 306, earbud 308, and a battery compartment 310. The ear-wearable stress monitoring system also includes a suggestion 1102.

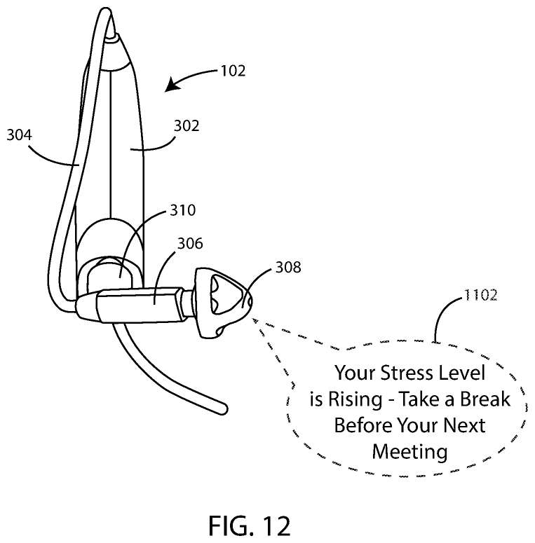

Referring now to FIG. 12, a schematic view of an ear-wearable device 102 is shown in accordance with various embodiments herein. The ear-wearable device 102 can be part of an ear-wearable stress monitoring system. The ear-wearable device 102 includes a housing 302, cable 304, receiver 306, earbud 308, and a battery compartment 310. The ear-wearable stress monitoring system also includes a suggestion 1102.

It will be appreciated that in some cases the trend regarding stress may be more important than an instantaneous measure or snapshot of stress. For example, an hour-long trend where detected stress rises to higher and higher levels may represent a greater health danger to an individual (and thus meriting intervention) than a brief spike in detected stress levels. As such, in various embodiments herein the ear-wearable stress monitoring system 700 wherein the ear-wearable stress monitoring system 700 is configured to record data regarding occurrences of stress and calculate a trend regarding the same. The trend can span minutes, hours, days, weeks or months. Various actions can be taken by the system or device in response to the trend. For example, wherein the trend is upward (a trend toward increased stress) the device may initiate suggestions for corrective actions and/or increase the frequency with which such suggestions are provided to the device wearer. If suggestions are already being provided and/or actions are already being taken by the device and the trend is upward (a negative trend toward increased stress) the device may be configured to change the suggestions/instructions being provided to the device wearer as the current suggestions/instructions are being empirically shown to be ineffective.

In various embodiments, the ear-wearable stress monitoring system can be configured to generate and/or use a predicted stress level of the device wearer in a subsequent time period. For example, in various embodiments, the ear-wearable stress monitoring system can be configured to cross-reference classified stress levels against a calendar of the device wearer and predict stress levels that may be reached during events upcoming on the calendar such as meetings. In some embodiments, the calendar information can be input into the system or device by the device wearer or another third party. In some embodiments, the calendar information can be supplied by an accessory device, such as a smart phone. In some embodiments, the calendar information can be retrieved using a calendar API. In some cases, the system can offer suggestions to the device wearer in order to prepare for such predicted episodes of stress.

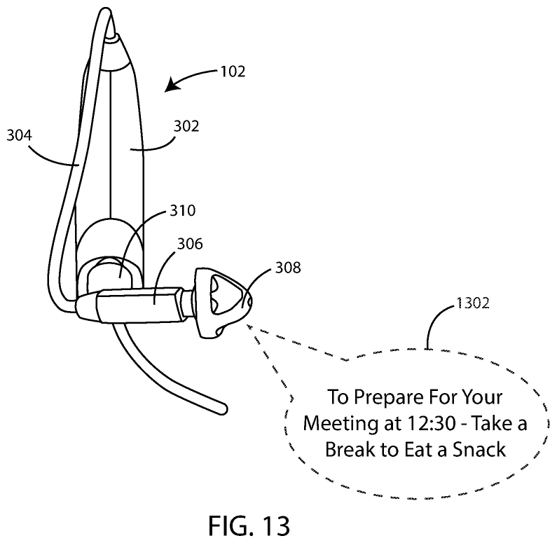

Referring now to FIG. 13, a schematic view of an ear-wearable device 102 is shown in accordance with various embodiments herein. The ear-wearable device 102 can be part of an ear-wearable stress monitoring system. The ear-wearable device 102 includes a housing 302, cable 304, receiver 306, earbud 308, and a battery compartment 310. The ear-wearable stress monitoring system also includes a predictive suggestion 1302.

In various embodiments, the ear-wearable stress monitoring system can be configured to execute an operation to remediate the trigger of the stress, independent of action of the device wearer. For example, many people find a quieter environment to be less stressful than the louder environment. In various embodiments, the ear-wearable stress monitoring system can execute an operation including reducing the volume of sounds exceeding a threshold value. In various embodiments, the ear-wearable stress monitoring system can reduce a volume sounds by actuating a vent or autovent. A vent or autovent feature of an ear-wearable device can be used to selectively seal off the ear canal creating acoustic separation from the ambient environment. Examples of vent features include, but are not limited to, those found in commonly owned U.S. patent application Ser. No. 13/720,793 (now issued as U.S. Pat. No. 8,923,543), entitled HEARING ASSISTANCE DEVICE VENT VALVE, and commonly-owned U.S. Provisional Patent Application No. 62/850,805, entitled SOLENOID ACTUATOR IN A HEARING DEVICE, both of which are hereby incorporated by reference herein in their entirety.

As another example of remediation or an action to mitigate stress, in various embodiments the ear-wearable stress monitoring system can execute an operation to reduce volume by adjusting a gain value of the ear-wearable stress monitoring system. As part of functionality associated with hearing assistance, devices herein can include amplifiers (digital or analog), filters (digital or analog), signal processing devices and the like. Operations of such devices can be altered to reduce a gain value so that sound provided to the device wearer via the ear-wearable device is reduced in volume. In this way, a sound that would otherwise be at a very high volume can be provided to the device wearer at a lesser volume, to reduce the impacts of the sound on stress and/or anxiety. In various embodiments, the ear-wearable stress and/or anxiety monitoring system can execute an operation to change noise processing or noise reduction features associated with the ear-wearable device or system. For example, some transient noise (such as sounds that might be particularly annoying or alarming) can be reduced or otherwise suppressed by modifying the noise reduction features of the system or device. This can include, for example, ambient noise reduction/cancellation, frequency specific noise reduction/cancellation, or the like. In some embodiments, other aspects of device operation and/or therapy provision can be changed by the system as part of an effort to remediate and/or mitigate stress. For example, tinnitus can be treated with an auditory stimulus and in embodiments herein the nature of the stimulus or parameters of the stimulus can be changed in order to mitigate stress. As a specific example, in some cases, the tinnitus stimulus can switch to music instead of noise (or vice versa if the device wearer prefers it) in order to help the device wearer reduce their stress level. Aspects of tinnitus therapy can be found in U.S. patent Ser. No. 10/537,268, the content of which is herein incorporated by reference.

In various embodiments, the ear-wearable stress monitoring system can be configured to correlate possible detected stressors with subsequent classified stress levels of the device wearer to elucidate cause and effect relationships. Then this data can be used in various ways. For example, in some embodiments, the ear-wearable stress monitoring system can be configured to weight certain possible detected stressors in the machine learning classification model more heavily based on an identified correlation between a particular stressor and resulting stress that holds true for the particular individual wearing the device. In some embodiments, such correlations can be used in order to predict future stress.

In various embodiments, the ear-wearable stress monitoring system can be configured to detect an occurrence of stress of the device wearer exceeding a threshold value. In various embodiments, the ear-wearable stress monitoring system can be configured to evaluate data from at least one of a microphone and a sensor package over a lookback period to detect a trigger of the stress exceeding a threshold value.

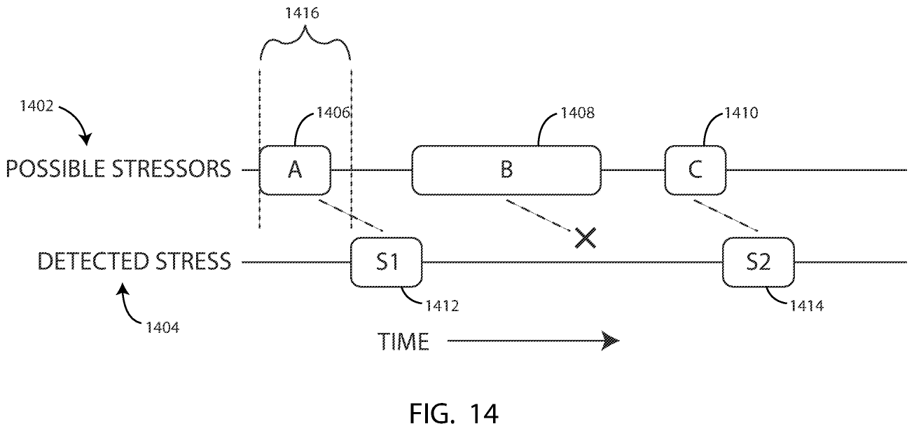

Referring now to FIG. 14, a schematic representation is shown of possible stressors 1402 and detected stress 1404 along a timeline. In this example, the possible stressors 1402 including stressor “A” 1406, “B” 1408, and “C” 1410. The detected stress 1404 includes stress episodes “S1” 1412 and “S2” 1414 that exceed a threshold value.

When a stress episode is detected, the system can evaluate data from at least one of a microphone and a sensor package over a lookback period 1416. In this example, stressor “A” 1406 falls within the lookback period and this can be taken as an indication that stressor “A” 1406 may be a possible stressor that actually results in a stress episode for the device wearer. To facilitate such operations, the device can be configured to store data for a rolling window of time reflecting the desired lookback period 1416.

In some embodiments, the lookback period 1416 can be greater than or equal to 5 seconds, 10 seconds, 30 seconds, 1 minute, 10 minutes, 20 minutes, 30 minutes, 40 minutes, 50 minutes, or 60 minutes, or can be an amount falling within a range between any of the foregoing.

In the example of FIG. 14, there is no detected stress episode within a lookback period of possible stressor “B” 1408, thus possible stressor “B” 1408 is not an actual stressor for the individual wearing the device. Further, stress episode S2 1414 appears to be linked with possible stressor “C” 1410 as it falls within the lookback period for stress episode S2 1414. In this manner, the device can determine which possible stressors act as actual stressors for the specific individual wearing the device and which do not, thereby customizing the monitoring, detection, and prediction capabilities of the system/device for the particular individual. For example, referring again to FIG. 14, if possible stressor “A” 1406 is observed (which could be any of the potential stressors described herein as well as others) or seen to be imminent (such as a meeting, travel to a specific location, meeting with a specific person, etc.) then the system can predict the onset of an episode of stress. As such, in some embodiments, the system can issue recommendations in advance (such as that illustrated in FIG. 13 or another type of recommendation) to help the device wearer be in the best position to handle the expected episode of stress.

In some embodiments, information regarding relationships between stress and possible stressors can be reported to the device wearer and/or to a third party. In some embodiments, relationships between episodes of stress and other aspects (such as a worsening of tinnitus) can be analyzed and/or reported to the device wearer and/or a third party. For example, in some individuals an increased stress level can cause a worsening of tinnitus, or vice-versa (a louder perceived tinnitus may increase anxiety/stress). The result of such an analysis can then be delivered to the user, caregivers, or hearing-care providers (e.g., “We noticed that your tinnitus gets worse when you are stressed. You should consider trying to avoid stressful situations.” As another example, “We noticed that you get anxious when your tinnitus becomes louder. Did you know that your hearing device can play sounds that temporarily mask your tinnitus?” if the device wearer is not already using such operational features.

Ear-wearable devices of the present disclosure can incorporate an antenna arrangement coupled to a high-frequency radio, such as a 2.4 GHz radio. The radio can conform to an IEEE 802.11 (e.g., WIFI®) or BLUETOOTH® (e.g., BLE, BLUETOOTH® 4. 2 or 5.0) specification, for example. It is understood that ear-wearable devices of the present disclosure can employ other radios, such as a 900 MHz radio or radios operating at other frequencies or frequency bands. Ear-wearable devices of the present disclosure can be configured to receive streaming audio (e.g., digital audio data or files) from an electronic or digital source. Representative electronic/digital sources (also referred to herein as accessory devices) include an assistive listening system, a TV streamer, a radio, a smartphone, a cell phone/entertainment device (CPED) or other electronic device that serves as a source of digital audio data or files. Systems herein can also include these types of accessory devices as well as other types of devices.

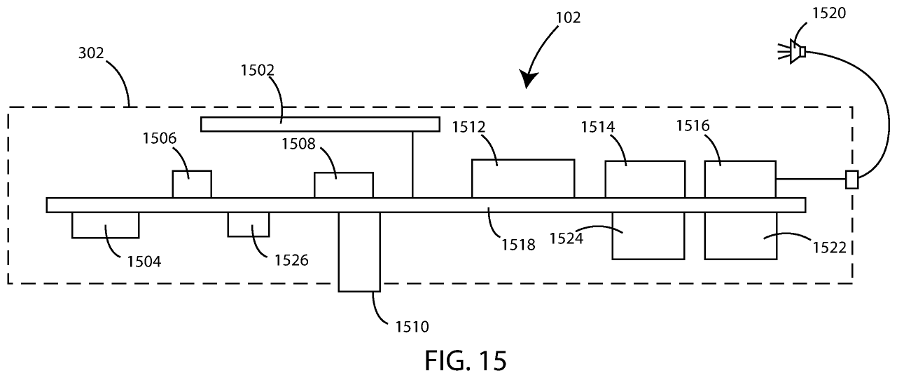

Referring now to FIG. 15, a schematic block diagram is shown with various components of an ear-wearable device in accordance with various embodiments. The block diagram of FIG. 15 represents a generic ear-wearable device for purposes of illustration. The ear-wearable device 102 shown in FIG. 15 includes several components electrically connected to a flexible mother circuit 1518 (e.g., flexible mother board) which is disposed within housing 302. A power supply circuit 1504 can include a battery and can be electrically connected to the flexible mother circuit 1518 and provides power to the various components of the ear-wearable device 102. One or more microphones 1506 are electrically connected to the flexible mother circuit 1518, which provides electrical communication between the microphones 1506 and a digital signal processor (DSP) 1512. Microphones herein can be of various types including, but not limited to, unidirectional, omnidirectional, MEMS based microphones, piezoelectric microphones, magnetic microphones, electret condenser microphones, and the like. Among other components, the DSP 1512 incorporates or is coupled to audio signal processing circuitry configured to implement various functions described herein. A sensor package 1514 can be coupled to the DSP 1512 via the flexible mother circuit 1518. The sensor package 1514 can include one or more different specific types of sensors such as those described in greater detail below. One or more user switches 1510 (e.g., on/off, volume, mic directional settings) are electrically coupled to the DSP 1512 via the flexible mother circuit 1518. It will be appreciated that the user switches 1510 can extend outside of the housing 302.

An audio output device 1516 is electrically connected to the DSP 1512 via the flexible mother circuit 1518. In some embodiments, the audio output device 1516 comprises a speaker (coupled to an amplifier). In other embodiments, the audio output device 1516 comprises an amplifier coupled to an external receiver 1520 adapted for positioning within an ear of a wearer. The external receiver 1520 can include an electroacoustic transducer, speaker, or loud speaker. The ear-wearable device 102 may incorporate a communication device 1508 coupled to the flexible mother circuit 1518 and to an antenna 1502 directly or indirectly via the flexible mother circuit 1518. The communication device 1508 can be a BLUETOOTH® transceiver, such as a BLE (BLUETOOTH® low energy) transceiver or other transceiver(s) (e.g., an IEEE 802.11 compliant device). The communication device 1508 can be configured to communicate with one or more external devices, such as those discussed previously, in accordance with various embodiments. In various embodiments, the communication device 1508 can be configured to communicate with an external visual display device such as a smart phone, a video display screen, a tablet, a computer, or the like.

In various embodiments, the ear-wearable device 102 can also include a control circuit 1522 and a memory storage device 1524. The control circuit 1522 can be in electrical communication with other components of the device. In some embodiments, a clock circuit 1526 can be in electrical communication with the control circuit. The control circuit 1522 can execute various operations, such as those described herein. In various embodiments, the control circuit 1522 can execute operations resulting in the provision of a user input interface by which the ear-wearable device 102 can receive inputs (including audible inputs, touch based inputs, and the like) from the device wearer. The control circuit 1522 can include various components including, but not limited to, a microprocessor, a microcontroller, an FPGA (field-programmable gate array) processing device, an ASIC (application specific integrated circuit), or the like. The memory storage device 1524 can include both volatile and non-volatile memory. The memory storage device 1524 can include ROM, RAM, flash memory, EEPROM, SSD devices, NAND chips, and the like. The memory storage device 1524 can be used to store data from sensors as described herein and/or processed data generated using data from sensors as described herein.

It will be appreciated that various of the components described in FIG. 15 can be associated with separate devices and/or accessory devices to the ear-wearable device. By way of example, microphones can be associated with separate devices and/or accessory devices. Similarly, audio output devices can be associated with separate devices and/or accessory devices to the ear-wearable device. Further accessory devices as discussed herein can include various of the components as described with respect to an ear-wearable device. For example, an accessory device can include a control circuit, a microphone, a motion sensor, and a power supply, amongst other things.

Accessory devices herein can include various different components. In some embodiments, the accessory device can be a personal communications device, such as a smart phone. However, the accessory device can also be other things such as a secondary wearable device, a handheld computing device, a dedicated location determining device (such as a handheld GPS unit), or the like.

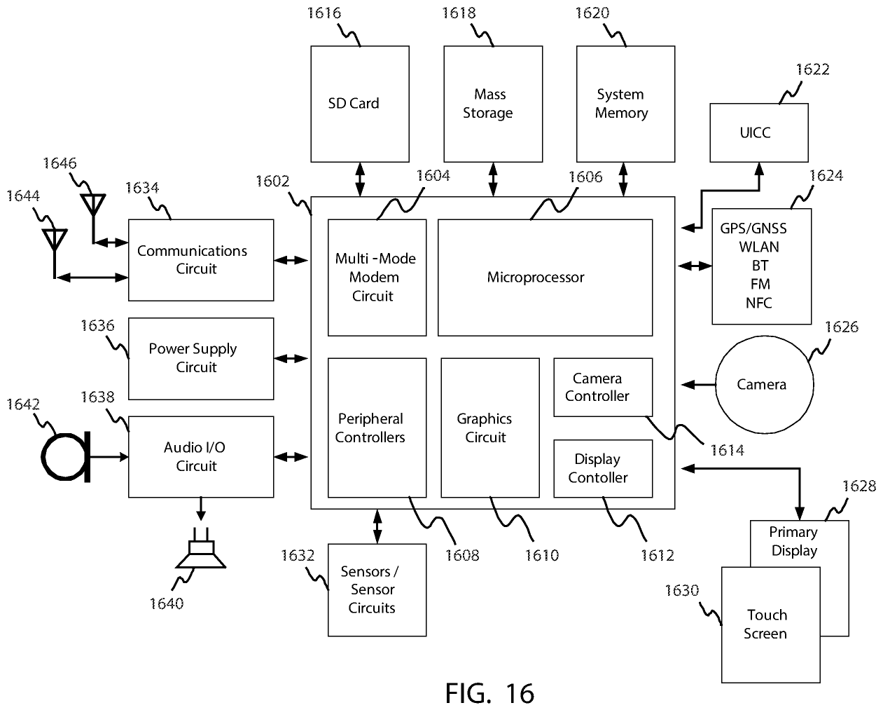

Referring now to FIG. 16, a schematic block diagram is shown of components of an accessory device (which could be a personal communications device or another type of accessory device) in accordance with various embodiments herein. This block diagram is just provided by way of illustration and it will be appreciated that accessory devices can include greater or lesser numbers of components. The accessory device in this example can include a control circuit 1602. The control circuit 1602 can include various components which may or may not be integrated. In various embodiments, the control circuit 1602 can include a microprocessor 1606, which could also be a microcontroller, FPGA, ASIC, or the like. The control circuit 1602 can also include a multi-mode modem circuit 1604 which can provide communications capability via various wired and wireless standards. The control circuit 1602 can include various peripheral controllers 1608. The control circuit 1602 can also include various sensors/sensor circuits 1632. The control circuit 1602 can also include a graphics circuit 1610, a camera controller 1614, and a display controller 1612. In various embodiments, the control circuit 1602 can interface with an SD card 1616, mass storage 1618, and system memory 1620. In various embodiments, the control circuit 1602 can interface with universal integrated circuit card (UICC) 1622. A spatial location determining circuit can be included and can take the form of an integrated circuit 1624 that can include components for receiving signals from GPS, GLONASS, BeiDou, Galileo, SBAS, WLAN, BT, FM, NFC type protocols, 5G picocells, or E911. In various embodiments, the accessory device can include a camera 1626. In various embodiments, the control circuit 1602 can interface with a primary display 1628 that can also include a touch screen 1630. In various embodiments, an audio I/O circuit 1638 can interface with the control circuit 1602 as well as a microphone 1642 and a speaker 1640. In various embodiments, a power supply or power supply circuit 1636 can interface with the control circuit 1602 and/or various other circuits herein in order to provide power to the system. In various embodiments, a communications circuit 1634 can be in communication with the control circuit 1602 as well as one or more antennas (1644, 1646).

Pattern Identification

It will be appreciated that in various embodiments herein, a device or a system can be used to detect a pattern or patterns indicative of an occurrence of a stress episode and/or a stress episode of a specific level of intensity. Such patterns can be detected in various ways. Some techniques are described elsewhere herein, but some further examples will now be described.

As merely one example, one or more sensors can be operatively connected to a controller (such as the control circuit described in FIG. 15) or another processing resource (such as a processor of another device or a processing resource in the cloud). The controller or other processing resource can be adapted to receive data representative of a characteristic of the subject from one or more of the sensors and/or determine statistics of the subject over a monitoring time period based upon the data received from the sensor. As used herein, the term “data” can include a single datum or a plurality of data values or statistics. The term “statistics” can include any appropriate mathematical calculation or metric relative to data interpretation, e.g., probability, confidence interval, distribution, range, or the like. Further, as used herein, the term “monitoring time period” means a period of time over which characteristics of the subject are measured and statistics are determined. The monitoring time period can be any suitable length of time, e.g., 1 millisecond, 1 second, 10 seconds, 30 seconds, 1 minute, 10 minutes, 30 minutes, 1 hour, etc., or a range of time between any of the foregoing time periods.

Any suitable technique or techniques can be utilized to determine statistics for the various data from the sensors, e.g., direct statistical analyses of time series data from the sensors, differential statistics, comparisons to baseline or statistical models of similar data, etc. Such techniques can be general or individual-specific and represent long-term or short-term behavior. These techniques could include standard pattern classification methods such as Gaussian mixture models, clustering as well as Bayesian approaches, machine learning approaches such as neural network models and deep learning, and the like.

Further, in some embodiments, the controller can be adapted to compare data, data features, and/or statistics against various other patterns, which could be prerecorded patterns (baseline patterns) of the particular individual wearing an ear-wearable device herein, prerecorded patterns (group baseline patterns) of a group of individuals wearing ear-wearable devices herein, one or more predetermined patterns that serve as patterns indicative of indicative of an occurrence of a stress episode (positive example patterns), one or more predetermined patterns that service as patterns indicative of the absence of a stress episode (negative example patterns), or the like. As merely one scenario, if a pattern is detected in an individual that exhibits similarity crossing a threshold value to a particular positive example pattern or substantial similarity to that pattern, wherein the pattern is specific for a stress episode and/or a stress episode of a specific level of intensity, then that can be taken as an indication of an occurrence of a stress episode experienced by the device wearer and/or a stress episode of a specific level of intensity experienced by the device wearer.

Similarity and dissimilarity can be measured directly via standard statistical metrics such normalized Z-score, or similar multidimensional distance measures (e.g., Mahalanobis or Bhattacharyya distance metrics), or through similarities of modeled data and machine learning. These techniques can include standard pattern classification methods such as Gaussian mixture models, clustering as well as Bayesian approaches, neural network models, and deep learning.

As used herein the term “substantially similar” means that, upon comparison, the sensor data are congruent or have statistics fitting the same statistical model, each with an acceptable degree of confidence. The threshold for the acceptability of a confidence statistic may vary depending upon the subject, sensor, sensor arrangement, type of data, context, condition, etc.

The statistics associated with the health status of an individual (and, in particular, their status with respect to stress), over the monitoring time period, can be determined by utilizing any suitable technique or techniques, e.g., standard pattern classification methods such as Gaussian mixture models, clustering, hidden Markov models, as well as Bayesian approaches, neural network models, and deep learning.

Various embodiments herein specifically include the application of a machine learning classification model. In various embodiments, the ear-wearable stress monitoring system can be configured to periodically update the machine learning classification model based on indicators of stress experienced by the device wearer and/or by observing stress experienced by the device wearer as caused by particular potential stressors.

In some embodiments, a training set of data can be used in order to generate a machine learning classification model. The input data can include microphone and/or sensor data as described herein as tagged/labeled with binary and/or non-binary classifications of stress. Binary classification approaches can utilize techniques including, but not limited to, logistic regression, k-nearest neighbors, decision trees, support vector machine approaches, naive Bayes techniques, and the like. Multi-class classification approaches (e.g., for non-binary classifications of stress) can include k-nearest neighbors, decision trees, naive Bayes approaches, random forest approaches, and gradient boosting approaches amongst others.

In various embodiments, the ear-wearable stress monitoring system is configured to execute operations to generate or update the machine learning model on the ear-wearable device itself. In some embodiments, the ear-wearable stress monitoring system may convey data to another device such as an accessory device or a cloud computing resource in order to execute operations to generate or update a machine learning model herein. In various embodiments, the ear-wearable stress monitoring system is configured to weight certain possible detected stressors in the machine learning classification model more heavily based on derived correlations specific for the individual as described elsewhere herein.

Sensor Package

Various embodiments herein include a sensor package. Specifically, systems and ear-wearable devices herein can include one or more sensor packages (including one or more discrete or integrated sensors) to provide data for use with operations to characterize the stress experienced by an individual as well as characterize possible stressors. Further details about the sensor package are provided as follows. However, it will be appreciated that this is merely provided by way of example and that further variations are contemplated herein. Also, it will be appreciated that a single sensor may provide more than one type of physiological data. For example, heart rate, respiration, blood pressure, or any combination thereof may be extracted from PPG sensor data.

In various embodiments, the indicators of stress experienced by the device wearer are derived from data produced by at least one of the microphone and the sensor package. In various embodiments, the sensor package can include at least one including at least one of a heart rate sensor, a heart rate variability sensor, an electrocardiogram (ECG) sensor, a blood oxygen sensor, a blood pressure sensor, a skin conductance sensor, a photoplethysmography (PPG) sensor, a temperature sensor (such as a core body temperature sensor, skin temperature sensor, ear-canal temperature sensor, or another temperature sensor), a motion sensor, an electroencephalograph (EEG) sensor, and a respiratory sensor. In various embodiments, the motion sensor can include at least one of an accelerometer and a gyroscope.

The sensor package can comprise one or a multiplicity of sensors. In some embodiments, the sensor packages can include one or more motion sensors (or movement sensors) amongst other types of sensors. Motion sensors herein can include inertial measurement units (IMU), accelerometers, gyroscopes, barometers, altimeters, and the like. The IMU can be of a type disclosed in commonly owned U.S. patent application Ser. No. 15/331,230, filed Oct. 21, 2016, which is incorporated herein by reference. In some embodiments, electromagnetic communication radios or electromagnetic field sensors (e.g., telecoil, NFMI, TMR, GMR, etc.) sensors may be used to detect motion or changes in position. In some embodiments, biometric sensors may be used to detect body motions or physical activity. Motions sensors can be used to track movements of a patient in accordance with various embodiments herein.

In some embodiments, the motion sensors can be disposed in a fixed position with respect to the head of a patient, such as worn on or near the head or ears. In some embodiments, the operatively connected motion sensors can be worn on or near another part of the body such as on a wrist, arm, or leg of the patient.

According to various embodiments, the sensor package can include one or more of an IMU, and accelerometer (3, 6, or 9 axis), a gyroscope, a barometer, an altimeter, a magnetometer, a magnetic sensor, an eye movement sensor, a pressure sensor, an acoustic sensor, a telecoil, a heart rate sensor, a global positioning system (GPS), a temperature sensor, a blood pressure sensor, an oxygen saturation sensor, an optical sensor, a blood glucose sensor (optical or otherwise), a galvanic skin response sensor, a cortisol level sensor (optical or otherwise), a microphone, acoustic sensor, an electrocardiogram (ECG) sensor, electroencephalography (EEG) sensor which can be a neurological sensor, eye movement sensor (e.g., electrooculogram (EOG) sensor), myographic potential electrode sensor (or electromyography—EMG), a heart rate monitor, a pulse oximeter or oxygen saturation sensor (SpO2), a wireless radio antenna, blood perfusion sensor, hydrometer, sweat sensor, cerumen sensor, air quality sensor, pupillometry sensor, cortisol level sensor, hematocrit sensor, light sensor, image sensor, and the like.

In some embodiments, the sensor package can be part of an ear-wearable device. However, in some embodiments, the sensor packages can include one or more additional sensors that are external to an ear-wearable device. For example, various of the sensors described above can be part of a wrist-worn or ankle-worn sensor package, or a sensor package supported by a chest strap. In some embodiments, sensors herein can be disposable sensors that are adhered to the device wearer (“adhesive sensors”) and that provide data to the ear-wearable device or another component of the system.

Data produced by the sensor(s) of the sensor package can be operated on by a processor of the device or system.

As used herein the term “inertial measurement unit” or “IMU” shall refer to an electronic device that can generate signals related to a body's specific force and/or angular rate. IMUs herein can include one or more accelerometers (3, 6, or 9 axis) to detect linear acceleration and a gyroscope to detect rotational rate. In some embodiments, an IMU can also include a magnetometer to detect a magnetic field.

The eye movement sensor may be, for example, an electrooculographic (EOG) sensor, such as an EOG sensor disclosed in commonly owned U.S. Pat. No. 9,167,356, which is incorporated herein by reference. The pressure sensor can be, for example, a MEMS-based pressure sensor, a piezo-resistive pressure sensor, a flexion sensor, a strain sensor, a diaphragm-type sensor and the like.

The temperature sensor can be, for example, a thermistor (thermally sensitive resistor), a resistance temperature detector, a thermocouple, a semiconductor-based sensor, an infrared sensor, or the like.

The blood pressure sensor can be, for example, a pressure sensor. The heart rate sensor can be, for example, an electrical signal sensor, an acoustic sensor, a pressure sensor, an infrared sensor, an optical sensor, or the like.

The oxygen saturation sensor (such as a blood oximetry sensor) can be, for example, an optical sensor, an infrared sensor, a visible light sensor, or the like.

The electrical signal sensor can include two or more electrodes and can include circuitry to sense and record electrical signals including sensed electrical potentials and the magnitude thereof (according to Ohm's law where V=IR) as well as measure impedance from an applied electrical potential.

It will be appreciated that the sensor package can include one or more sensors that are external to the ear-wearable device. In addition to the external sensors discussed hereinabove, the sensor package can comprise a network of body sensors (such as those listed above) that sense movement of a multiplicity of body parts (e.g., arms, legs, torso). In some embodiments, the ear-wearable device can be in electronic communication with the sensors or processor of another medical device, e.g., an insulin pump device or a heart pacemaker device.

Methods

Many different methods are contemplated herein, including, but not limited to, methods of making, methods of using, methods of detecting stress or anxiety, methods of detecting stressors, methods of monitoring stress or anxiety, methods of treating stress or anxiety, or preventing stress or anxiety, and the like. Aspects of system/device operation described elsewhere herein can be performed as operations of one or more methods in accordance with various embodiments herein.

In an embodiment, a method of monitoring stress or anxiety of an individual using an ear-wearable stress monitoring system is included, the method including evaluating data from at least one of a microphone and a sensor package, classifying a stress level of the individual using a machine learning classification model, and updating the machine learning classification model based on indicators of stress experienced by the individual.

In an embodiment, the method can further include deriving indicators of stress experienced by the device wearer from data produced by at least one of the microphone and the sensor package. In an embodiment, the method can further include deriving indicators of stress experienced by the device wearer from input provided by the individual. In an embodiment, the method can further include deriving indicators of stress experienced by the device wearer from input provided by an external device.

In an embodiment, the method can further include correlating possible detected stressors with subsequent classified stress levels of the device wearer. In an embodiment of the method, correlating possible detected stressors with subsequent classified stress levels of the device wearer further comprises weighting certain possible detected stressors in the machine learning classification model more heavily based on the correlation.

In an embodiment, the method can further include processing data from the microphone to distinguish a voice of the individual from a voice of a third party.

In an embodiment, the method can further include detecting meals of the individual and correlating classified stress levels with detected meals.

In an embodiment, the method can further include detecting displacement behaviors and correlating classified stress levels with detected displacement behaviors.

In an embodiment, the method can further include sending information regarding classified stress levels to an electronic medical record system. In an embodiment, the method can further include sending information regarding classified stress levels to a third party.

In an embodiment, the method can further include distinguishing between acute and chronic stress of the device wearer.

In an embodiment, a method of monitoring stress of an individual using an ear-wearable stress monitoring system is included, the method including evaluating data from at least one of a microphone and a sensor package, classifying a stress level of the individual using a machine learning classification model, and providing information to the individual relating to the classified stress level in a discrete manner.

In an embodiment, the method can further include providing information to the individual relating to the classified stress level through an electroacoustic transducer in the ear canal of the individual.

In an embodiment, the method can further include providing instructions to the individual regarding an action to take to reduce the stress level of the individual. In an embodiment of the method, the instructions to the individual comprise breathing instructions. In an embodiment of the method, the instructions to the individual comprise a suggestion to consume a food item.

In an embodiment of the method, information to the individual relating to the classified stress level comprises a sound preselected by the device wearer.

In an embodiment of the method, information to the individual relating to the classified stress level includes information regarding a predicted stress level of the device wearer in a subsequent time period.

In an embodiment, the method can further include deriving indicators of stress experienced by the device wearer from input provided by an external device.

In an embodiment, the method can further include correlating possible detected stressors with subsequent classified stress levels of the device wearer. In an embodiment of the method, correlating possible detected stressors with subsequent classified stress levels of the device wearer further comprises weighting certain possible detected stressors in the machine learning classification model more heavily based on the correlation.

In an embodiment, a method of monitoring stress of an individual using an ear-wearable stress monitoring system is included, the method including detecting an occurrence of stress of the device wearer exceeding a threshold value and evaluating data from at least one of a microphone and a sensor package over a lookback period to detect a trigger of the stress.

In an embodiment, the method can further include executing an operation to remediate the trigger of the stress. In an embodiment of the method, the operation comprises reducing the volume of sounds exceeding a threshold value. In an embodiment of the method, the volume is reduced by adjusting a gain value of the ear-wearable device. In an embodiment of the method, the volume is decreased by actuating an autovent.

In an embodiment, the method can further include providing information to the individual relating to the classified stress level through an electroacoustic transducer in the ear canal of the individual. In an embodiment, the method can further include providing instructions to the individual regarding an action to take related to the detected trigger of the stress. In an embodiment of the method, the instructions to the individual comprise breathing instructions. In an embodiment of the method, the instructions to the individual comprise a suggestion to consume a food item and/or take a medication

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. An ear-wearable stress and/or anxiety monitoring system comprising:
a control circuit;
a microphone, wherein the microphone is in electrical communication with the control circuit; and
a sensor package, the sensor package comprising a motion sensor;
wherein the sensor package is in electrical communication with the control circuit;
wherein the ear-wearable system is configured to
evaluate data from at least one of the microphone and the sensor package and classify a stress level of a device wearer using a machine learning classification model; and
periodically update the machine learning classification model based on indicators of stress experienced by the device wearer.

2. The ear-wearable system of any of claims 1 and 3-22, wherein the indicators of stress experienced by the device wearer are derived from data produced by at least one of the microphone and the sensor package.

3. The ear-wearable system of any of claims 1-2 and 4-22, wherein the indicators of stress experienced by the device wearer are derived from inputs provided by the device wearer.

4. The ear-wearable system of any of claims 1-3 and 5-22, wherein the inputs provided by the device wearer comprise a tap.

5. The ear-wearable system of any of claims 1-4 and 6-22, wherein the inputs provided by the device wearer comprise a head gesture.

6. The ear-wearable system of any of claims 1-5 and 7-22, wherein the inputs provided by the device wearer comprise a response to a query.

7. The ear-wearable system of any of claims 1-6 and 8-22, wherein the indicators of stress experienced by the device wearer are received by the ear-wearable system from an external source.

8. The ear-wearable system of any of claims 1-7 and 9-22, wherein the ear-wearable system is configured to correlate possible detected stressors with subsequent classified stress levels of the device wearer.

9. The ear-wearable system of any of claims 1-8 and 10-22, wherein the ear-wearable system is configured to weight certain possible detected stressors in the machine learning classification model more heavily based on the correlation.

10. The ear-wearable system of any of claims 1-9 and 11-22, wherein data from the microphone is processed to distinguish a voice of the device wearer from a voice of a third party.

11. The ear-wearable system of any of claims 1-10 and 12-22, wherein the voice of the device wearer is treated as an indicator of stress experienced by the device wearer.

12. The ear-wearable system of any of claims 1-11 and 13-22, wherein the voice of the third party is treated as a possible detected stressor.

13. The ear-wearable system of any of claims 1-12 and 14-22, wherein the ear-wearable system is configured to correlate sleep of the device wearer with subsequent classified stress levels of the device wearer.

14. The ear-wearable system of any of claims 1-13 and 15-22, wherein the ear-wearable system is configured to detect meals and correlate classified stress levels with detected meals.

15. The ear-wearable system of any of claims 1-14 and 16-22, wherein the ear-wearable system is configured to correlate stress levels with a time since a last meal.

16. The ear-wearable system of any of claims 1-15 and 17-22, wherein the ear-wearable system is configured to detect displacement behaviors and correlate classified stress levels with detected displacement behaviors.

17. The ear-wearable system of any of claims 1-16 and 18-22, the sensor package comprising at least one selected from the group consisting of a heart rate sensor, a heart rate variability sensor, an electrocardiogram (ECG) sensor, a blood oxygen sensor, a blood pressure sensor, a skin conductance sensor, a photoplethysmography (PPG) sensor, an electromyography (EMG) sensor, a core body temperature sensor, a motion sensor, an electroencephalograph (EEG) sensor, and a respiratory sensor.

18. The ear-wearable system of any of claims 1-17 and 19-22, wherein the motion sensor comprises at least one of an accelerometer and a gyroscope.

19. The ear-wearable system of any of claims 1-18 and 20-22, wherein the ear-wearable system is configured to send information regarding classified stress levels to an electronic medical record system.

20. The ear-wearable system of any of claims 1-19 and 21-22, wherein the ear-wearable system is configured to send information regarding classified stress levels to a third party.

21. The ear-wearable system of any of claims 1-20 and 22, wherein the ear-wearable system is configured to distinguish between acute and chronic stress of the device wearer.

22. The ear-wearable system of any of claims 1-21, wherein the ear-wearable system is configured to classify detected stress as acute stress, chronic stress, or both.

23. A method of monitoring stress of an individual using an ear-wearable stress and/or anxiety monitoring system comprising:
evaluating data from at least one of a microphone and a sensor package;
classifying a stress level of the individual using a machine learning classification model; and
updating the machine learning classification model based on indicators of stress experienced by the individual.

24. The method of any of claims 23 and 25-34, further comprising deriving indicators of stress experienced by the individual from data produced by at least one of the microphone and the sensor package.

25. The method of any of claims 23-24 and 26-34, further comprising deriving indicators of stress experienced by the individual from input provided by the individual.

26. The method of any of claims 23-25 and 27-34, further comprising deriving indicators of stress experienced by the individual from input provided by an external device.

27. The method of any of claims 23-26 and 28-34, further comprising correlating possible detected stressors with subsequent classified stress levels of the individual.

28. The method of any of claims 23-27 and 29-34, wherein correlating possible detected stressors with subsequent classified stress levels of the individual further comprises weighting certain possible detected stressors in the machine learning classification model more heavily based on the correlation.

29. The method of any of claims 23-28 and 30-34, further comprising processing data from the microphone to distinguish a voice of the individual from a voice of a third party.

30. The method of any of claims 23-29 and 31-34, further comprising detecting meals of the individual and correlating classified stress levels with detected meals.

31. The method of any of claims 23-30 and 32-34, further comprising detecting displacement behaviors and correlating classified stress levels with detected displacement behaviors.

32. The method of any of claims 23-31 and 33-34, further comprising sending information regarding classified stress levels to an electronic medical record system.

33. The method of any of claims 23-32 and 34, further comprising sending information regarding classified stress levels to a third party.

34. The method of any of claims 23-33, further comprising distinguishing between acute and chronic stress of the individual.

35. An ear-wearable stress and/or anxiety monitoring system comprising:
a control circuit;
a microphone, wherein the microphone is in electrical communication with the control circuit;
an electroacoustic transducer, wherein the electroacoustic transducer is configured for placement within an ear canal of a device wearer; and
a sensor package, the sensor package comprising a motion sensor;
wherein the sensor package is in electrical communication with the control circuit;
wherein the ear-wearable system is configured to
evaluate data from at least one of the microphone and the sensor package and classify a device wearer's stress level using a machine learning classification model; and
provide information to the device wearer relating to the classified stress level in a discrete manner.

36. The ear-wearable system of any of claims 35 and 37-51, wherein the information relating to the classified stress level is provided through the electroacoustic transducer.

37. The ear-wearable system of any of claims 35-36 and 38-51, wherein the information provided to the device wearer relating the classified stress level comprises verbal information.

38. The ear-wearable system of any of claims 35-37 and 39-51, wherein the information provided to the device wearer relating the classified stress level comprises non-verbal sound.

39. The ear-wearable system of any of claims 35-38 and 40-51, wherein the non-verbal sound comprises music.

40. The ear-wearable system of any of claims 35-39 and 41-51, wherein the information relating to the classified stress level includes instructions for an action for the device wearer to take to reduce the stress level of the device wearer.

41. The ear-wearable system of any of claims 35-40 and 42-51, wherein the instructions comprise breathing instructions.

42. The ear-wearable system of any of claims 35-41 and 43-51, wherein the breathing instructions comprise a suggestion to consume a food item and/or take a medication.

43. The ear-wearable system of any of claims 35-42 and 44-51, wherein the information relating to the classified stress level includes a sound preselected by the device wearer.

44. The ear-wearable system of any of claims 35-43 and 45-51, wherein the information relating to the classified stress level includes information regarding a predicted stress level of the device wearer in a subsequent time period.

45. The ear-wearable system of any of claims 35-44 and 46-51, wherein the ear-wearable system is configured to periodically update the machine learning classification model based on indicators of stress experienced by the device wearer.

46. The ear-wearable system of any of claims 35-45 and 47-51, further comprising a power supply circuit.

47. The ear-wearable system of any of claims 35-46 and 48-51, wherein the ear-wearable system is configured to send information regarding classified stress levels to an electronic medical record system.

48. The ear-wearable system of any of claims 35-47 and 49-51, wherein the ear-wearable system is configured to send information regarding classified stress levels to a third party.

49. The ear-wearable system of any of claims 35-48 and 50-51, wherein the ear-wearable system is configured to cross-reference classified stress levels against a calendar of the device wearer.

50. The ear-wearable system of any of claims 35-49 and 51, wherein the ear-wearable system is configured to distinguish between acute and chronic stress of the device wearer.

51. The ear-wearable system of any of claims 35-50, wherein the ear-wearable system is configured to detect meals of the device wearer and cross-reference classified stress levels against detected meal times.

52. A method of monitoring stress of an individual using an ear-wearable stress and/or anxiety monitoring system comprising:
evaluating data from at least one of a microphone and a sensor package;
classifying a stress level of the individual using a machine learning classification model; and
providing information to the individual relating to the classified stress level in a discrete manner.

53. The method of any of claims 52 and 54-67, further comprising providing information to the individual relating to the classified stress level through an electroacoustic transducer in the ear canal of the individual.

54. The method of any of claims 52-53 and 55-67, further comprising providing instructions to the individual regarding an action to take to reduce the stress level of the individual.

55. The method of any of claims 52-54 and 56-67, wherein the instructions to the individual comprise breathing instructions.

56. The method of any of claims 52-55 and 57-67, wherein the instructions to the individual comprise a suggestion to consume a food item and/or take a medication.

57. The method of any of claims 52-56 and 58-67, wherein information to the individual relating to the classified stress level comprises a sound preselected by the device wearer.

58. The method of any of claims 52-57 and 59-67, wherein information to the individual relating to the classified stress level includes information regarding a predicted stress level of the device wearer in a subsequent time period.

59. The method of any of claims 52-58 and 60-67, further comprising deriving indicators of stress experienced by the individual from input provided by an external device.

60. The method of any of claims 52-59 and 61-67, further comprising correlating possible detected stressors with subsequent classified stress levels of the individual.

61. The method of any of claims 52-60 and 62-67, wherein correlating possible detected stressors with subsequent classified stress levels of the individual further comprises weighting certain possible detected stressors in the machine learning classification model more heavily based on the correlation.

62. The method of any of claims 52-61 and 63-67, further comprising processing data from the microphone to distinguish a voice of the individual from a voice of a third party.

63. The method of any of claims 52-62 and 64-67, further comprising detecting meals of the individual and correlating classified stress levels with detected meals.

64. The method of any of claims 52-63 and 65-67, further comprising detecting displacement behaviors and correlating classified stress levels with detected displacement behaviors.

65. The method of any of claims 52-64 and 66-67, further comprising sending information regarding classified stress levels to an electronic medical record system.

66. The method of any of claims 52-65 and 67, further comprising sending information regarding classified stress levels to a third party.

67. The method of any of claims 52-66, further comprising distinguishing between acute and chronic stress of the individual.

68. An ear-wearable stress and/or anxiety monitoring system comprising:
a control circuit;
a microphone, wherein the microphone is in electrical communication with the control circuit;
an electroacoustic transducer, wherein the electroacoustic transducer is configured for placement within an ear canal of a device wearer; and
a sensor package, the sensor package comprising a motion sensor;
wherein the sensor package is in electrical communication with the control circuit;
wherein the ear-wearable system is configured to
detect an occurrence of stress of the device wearer exceeding a threshold value; and
evaluate data from at least one of the microphone and the sensor package over a lookback period to detect a trigger or contributing cause of the stress.

69. The ear-wearable system of any of claims 68 and 70-80, wherein the ear-wearable system is configured to execute an operation to remediate the trigger or contributing cause of the stress.

70. The ear-wearable system of any of claims 68-69 and 71-80, wherein the operation comprises reducing the volume of sounds exceeding a threshold value.

71. The ear-wearable system of any of claims 68-70 and 72-80, wherein the volume is reduced by actuating an autovent.

72. The ear-wearable system of any of claims 68-71 and 73-80, wherein the volume is reduced by adjusting a gain value of the ear-wearable system.

73. The ear-wearable system of any of claims 68-72 and 74-80, wherein the ear-wearable system is further configured to characterize the stress causing potential of ambient sound.

74. The ear-wearable system of any of claims 68-73 and 75-80, wherein the ear-wearable system is configured to provide instructions regarding recommended interventions to the device wearer.

75. The ear-wearable system of any of claims 68-74 and 76-80, wherein the ear-wearable system is configured to record data regarding occurrences of stress and calculate a trend regarding the same.

76. The ear-wearable system of any of claims 68-75 and 77-80, wherein the ear-wearable system is configured to change instructions regarding recommended interventions if the trend is negative.

77. The ear-wearable system of any of claims 68-76 and 78-80, wherein the ear-wearable system is configured to predict a future occurrence of stress of the device wearer based on at least one of current inputs from the microphone and/or sensor package, information from a calendar of the device wearer, and a geographic location of the device wearer.

78. The ear-wearable system of any of claims 68-77 and 79-80, wherein the ear-wearable system is configured to distinguish between acute and chronic stress of the device wearer.

79. The ear-wearable system of any of claims 68-78 and 80, wherein the ear-wearable system is configured to evaluate data from the microphone to identify a voice of an individual serving as a trigger of the stress.

80. The ear-wearable system of any of claims 68-79, wherein the ear-wearable system is configured to evaluate data received from a personal electronic device of a third person to identify an individual serving as a trigger of the stress.

81. A method of monitoring stress and/or anxiety of an individual using an ear-wearable stress monitoring system comprising:
detecting an occurrence of stress of the device wearer exceeding a threshold value; and
evaluating data from at least one of a microphone and a sensor package over a lookback period to detect a trigger of the stress.

82. The method of any of claims 81 and 83-96, further comprising executing an operation to remediate the trigger of the stress.

83. The method of any of claims 81-82 and 84-96, wherein the operation comprises reducing the volume of sounds exceeding a threshold value.

84. The method of any of claims 81-83 and 85-96, wherein the volume is reduced by adjusting a gain value.

85. The method of any of claims 81-84 and 86-96, wherein the volume is decreased by actuating an autovent.

86. The method of any of claims 81-85 and 87-96, further comprising providing information to the individual relating to the classified stress level through an electroacoustic transducer in the ear canal of the individual.

87. The method of any of claims 81-86 and 88-96, further comprising providing instructions to the individual regarding an action to take related to the detected trigger of the stress.

88. The method of any of claims 81-87 and 89-96, wherein the instructions to the individual comprise breathing instructions.

89. The method of any of claims 81-88 and 90-96, wherein the instructions to the individual comprise a suggestion to consume a food item and/or take a medication.

90. The method of any of claims 81-89 and 91-96, further comprising deriving indicators of stress experienced by the individual from input provided by an external device.

91. The method of any of claims 81-90 and 92-96, further comprising correlating possible detected stressors with subsequent classified stress levels of the individual.

92. The method of any of claims 81-91 and 93-96, wherein correlating possible detected stressors with subsequent classified stress levels of the individual further comprises weighting certain possible detected stressors in the machine learning classification model more heavily based on the correlation.

93. The method of any of claims 81-92 and 94-96, further comprising processing data from a microphone to distinguish a voice of the individual from a voice of a third party.

94. The method of any of claims 81-93 and 95-96, further comprising sending information regarding detected stress triggers to an electronic medical record system.

95. The method of any of claims 81-94 and 96, further comprising sending information regarding detected stress triggers to a third party.

96. The method of any of claims 81-95, further comprising identifying a voice of another individual serving as a detected stress trigger.
